(19) United States
(12) Patent Application Publication
(10) Pub. No.: US 2010/0297180 A1
(43) Pub. Date: Nov. 25, 2010

(54) BOTULINUM NEUROTOXIN VACCINE

(76) Inventor: Clifford Shone, Wiltshire (GB)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/682,544

(22) PCT Filed: Oct. 13, 2008
(86) PCT No.: PCT/GB08/50937

§ 371 Date: Aug. 9, 2010

Related U.S. Application Data
(60) Provisional application No. 61/006,546, filed on Jan. 18, 2008.
(60) Provisional application No. 60/960,771, filed on Oct. 12, 2007.

(30) Foreign Application Priority Data
Jan. 18, 2008 (GB) 0800927.6

(51) Int. Cl. A61K 39/08; A61P 37/04; A61P 31/04

(52) U.S. Cl. 424/239.1

(57) ABSTRACT

The invention provides compositions comprising an antigenic botulinum neurotoxin serotype E (BoNT/E) peptide, wherein the peptide has improved solubility and thus improved ability to stimulate an immune response against BoNT/E holotoxin. The composition may be used as part of a multivalent vaccine regimen via coordinated use with non-serotype E BoNT peptides, such as one or more BoNT/A and/or BoNT/B peptides. It further provides processes for manufacturing said BoNT/E peptide. It also provides methods of stimulating an immune response in a mammal, comprising administering to the mammal an effective amount of a BoNT/E and optionally one or more non-serotype E BoNT peptides such as one or more BoNT/A and/or BoNT/B peptides.

### FIELD OF THE INVENTION

The present invention relates to compositions and/or vaccines providing protection against botulinum neurotoxin serotype E (BoNT/E).

### BACKGROUND

The botulinum neurotoxins are a family of seven antigenically different protein toxins (serotypes A-G). These neurotoxins are extremely potent neuroparalytic agents which act primarily at the peripheral nervous system where they inhibit the release of acetylcholine at the neuromuscular junction (Niemann (1991) in Sourcebook of Bacterial Protein Toxins (Alouf, J. E. & Freer, J. H. eds.) pp. 303-348). Academic Press, London). This is mediated via highly specific zinc-dependent endopeptidase activity directed at small proteins involved in the fusion and release of synaptic vesicles. The botulinum neurotoxins (BoNTs) are structurally similar; they have 30-40% sequence homology and, as diagrammatically shown immediately below, each neurotoxin consists of a heavy chain (100 kDa) and a light chain (50 kDa) linked by a disulphide bridge (Niemann, 1991, as above). Despite structural similarities, antisera raised against purified neurotoxins show no cross-protection between the neurotoxin serotypes and thus necessitate the development of a separate vaccine for each serotype. In addition, within each serotype, various subtypes exist (Minton (1995) in Current Topics in Microbiology and Immunology 195 ‘Clostridial Neurotoxins’ (Montecucco, C., ed.) pp. 161-194, Springer, Berlin). Since these subtypes differ in their antigenic properties, the presence of these toxin variants needs to be taken into account in vaccine design to ensure adequate cross protection.

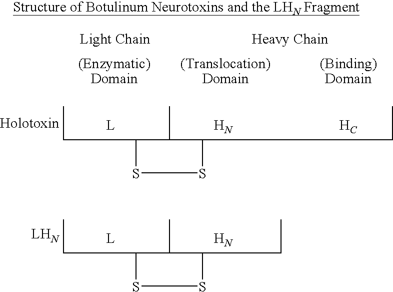

The C-terminal 50 kDa fragment (H_{C} fragment) is responsible for receptor-binding at the presynaptic nerve surface (Halpern & Loftus (1993) J. Biol. Chem. 268, 11188-11192); (Shone et al. (1985) Eur. J. Biochem., 151, 75-82). The N-terminal 50 kDa portion of the heavy chain (H_{N} fragment) is involved in translocation of the enzymatically active light chain to within the nerve terminal (Shone et al. (1987) Eur. J. Biochem., 167, 175-180). Removal of the H_{C} domain from the BoNT leaves a fragment (LH_{N}) consisting of the light chain and translocation domain which, although virtually non-toxic, is stable and soluble.

Many toxins require inactivation before they can be used as vaccines and chemical modifying agents such as formaldehyde have been widely used in vaccine production. Notable examples include: tetanus, diphtheria, botulinum and anthrax vaccines. Protein modification by formaldehyde is complex and involves the chemical modification of several amino acid residues and also the formation of cross-links, which can lead to extensive protein aggregation (Metz et al. (2004) J. Biol. Chem., 279: 6235-6243)

The use of formaldehyde for vaccine production has drawbacks, the most common of which is the modification of protein structure which results in a loss of immunogenic epitopes and an over all decrease in the immunogenicity of the protein (Vani et al. (2006) J. Immunol. Methods. 317, 80-89).

Current, second generation botulinum vaccines are based on non-toxic fragments of the botulinum toxins and are designed to eliminate the requirement for a detoxification step with formaldehyde. However, a common problem with second generation vaccines is that the recombinant polypeptides are poorly soluble (or even insoluble).

Botulinum type E neurotoxins can be divided into 4 subtypes based on amino acid sequence. These subtypes are labelled E₁, E₂, E₃ and E_{butyricum} and are closely related with regard to amino acid sequence displaying a maximum of 5% heterology (Hill et al. (2007) J. bacterial. 189: 818-832).

The production of a recombinant vaccine for botulinum type E has proved problematic. Type E vaccines based on the receptor binding domain (the H_{C} fragment) are unstable at physiological temperature and provide poor protective efficacy after a single dose. In addition, vaccines based on the LH_{N} fragment of type E neurotoxin have proved problematic. For example, as explained in WO 2007/044382 published 19 Apr. 2007, expression in E. coli of LH_{N} fragments from sub-type E₁ (e.g. from Clostridium botulinum strain beluga) have resulted in insoluble, mis-folded products, which do not provide a protective immune response against type E neurotoxin.

There is therefore a need for improved botulinum antigenic compositions/vaccines containing antigenic peptides having improved solubility and/or botulinum compositions/vaccines having improved protective immune response against BoNT, especially against BoNT/E. Linked to this prior art problem, there is also a need for an antigenic composition/vaccine having protection against multiple BoNT subtypes (within a given serotype, especially BoNT/E) and/or having protection against more than one BoNT serotype (preferably including BoNT/E).

The present invention meets this need by providing an antigenic composition/vaccine that solves one or more of the above problems.

In more detail, the present invention provides an antigenic composition/vaccine, comprising a botulinum serotype E (BoNT/E) peptide, said peptide comprising an amino acid sequence having at least 90% sequence identity to the peptide sequence provided by amino acid residues 100-750 of SEQ ID NO: 1:
(i) wherein the BoNT/E peptide contains one or more of H142, G176, S198, I199, T230, C231, I232, Q236, R244, K245, I247, N258, V265, Y268, N277, R280, Q295, I302, Q304, E305, D325, L328, A340, E349, K397, and L773; and
(i) wherein said BoNT/E peptide lacks a functional H_{C} of a clostridial neurotoxin heavy chain.

In one embodiment, the antigenic composition/vaccine comprises a BoNT/E peptide comprising (or consisting of) an amino acid sequence having 90, 92, 94, 96, 98, or 100% sequence identity to the peptide sequence provided by amino acid residues 100-750 of SEQ ID NO: 1.

In one embodiment, the antigenic composition/vaccine comprises a BoNT/E peptide comprising (or consisting of) an amino acid sequence having 90, 92, 94, 96, 98, or 100% sequence identity to the peptide sequence provided by amino acid residues 100-773 of SEQ ID NO: 1.

In one embodiment, the antigenic composition/vaccine comprises a BoNTIE peptide comprising (or consisting of) an amino acid sequence having 90, 92, 94, 96, 98, or 100% sequence identity to an amino acid sequence from amino acid residue 10, 20, 30, 40, 50, 60, 70, 80, 90 or 100 of SEQ ID NO: 1 to amino acid residue 750, 760, 770, 780, 790, 800, 810, 820, 830 or 840 of SEQ ID NO: 1.

In one embodiment, the antigenic composition/vaccine comprises a BoNT/E peptide comprising (or consisting of) an amino acid sequence having 90, 92, 94, 96, 98, or 100% sequence identity to the peptide sequence provided by amino acid residues 1-844 of SEQ ID NO: 1.

Also provided, is use of the above BoNT/E peptide for stimulating an immune response in an animal. The BoNT/E peptide of the invention provides (simultaneous) protection against challenge from one or more different BoNT/E subtypes, notably BoNT/E₃, BoNT/E₁ and/or BoNT/E₂. In addition, when combined with one or more of BoNT peptide(s) from a serotype other than BoNT/E (e.g. BoNT/A and/or BoNT/B), the present invention provides multivalent protection against BoNT/E and the non-E BoNT serotype(s).

The invention further provides a nucleic acid encoding the above BoNT/E peptide, together with use of said nucleic acid for expressing said BoNT/E peptide.

### DETAILED DESCRIPTION OF THE INVENTION

The peptide of the present invention comprises a botulinum neurotoxin (BoNT/E) LH_{N} fragment (including highly homologous sequence variants thereof). Thus, peptides of the present invention comprise a portion of a BoNT, which equates approximately to the N-terminal two-thirds of a BoNT holotoxin. The peptides are preferably in a single chain form (though may separately be in a di-chain ‘activated’ form), and lack the ability of the parent BoNT holotoxin to bind to nerve endings.

In one embodiment, a peptide of the present invention has reduced toxicity (eg. the peptide is substantially non-toxic) as compared with a naturally-occurring BoNT/E. In general, it is preferred to reduce (or eliminate) the toxicity of a peptide that is for administration to an individual (eg. as a therapeutic).

In one embodiment, the BoNT/E peptide of the invention is at least 75% less toxic than BoNT/E holotoxin, such as at least 80, 90, 95, 99 or 100% less toxic. In one embodiment, the BoNT/E peptide of the invention exhibits less than 25% of the toxicity of BoNT/E holotoxin, such as less than 20, 15, 10, 5 or 1% of the toxicity.

A number of conventional tests (such as a mouse bioassay) are available to determine the toxicity of a fusion protein.

In one embodiment of the invention, a vaccine/antigenic composition is provided based on an LH_{N} fragment of a BoNT/E (which consists of the light chain and N-terminal 50 kDa of heavy chain).

In another embodiment of the invention, a vaccine/antigenic composition is provided that includes a peptide comprising an extended LH_{N} fragment of a BoNT/E, though still lacking a functional H_{C} binding domain of BoNT. By way of example, the BoNT/E peptide may be extended (C-terminally) into the H_{C} portion of BoNT/E. One example of a H_{C} portion of BoNT/E is provided by SEQ ID NO: 7 (or an amino acid sequence having at least 90%, 92%, 94%, 96% 98% or 100% sequence identity thereto).

In one embodiment, the extended LH_{N} BoNT/E peptide comprises at least 25, 50, 100, 200, 250, 300, 350 or 400 consecutive amino acids of a BoNT/E heavy chain H_{C} portion (eg. SEQ ID NO: 7). In one embodiment, the extended LH_{N} BoNT/E peptide comprises less than about 50, 100, 200, 250, 300, 350, 400 or 425 consecutive amino acids of a BoNT heavy chain H_{C} portion (eg. SEQ ID NO: 7).

The extended LH_{N} BoNT/E peptide may extend into the BoNT/E H_{C} portion by any number of amino acids. For example, the BoNT/E peptide of the present invention may include (starting from the N-terminus of H_{C}) the first 10, 20, 30, 40, 50, 60, 70 or 80 amino acids of a BoNT/E H_{C} portion.

Thus, in one embodiment, the extended LH_{N} BoNT/E peptide may extend into the BoNT/E H_{C} portion up to amino acid residue 10, 20, 30, 40, 50, 60, 70 or 80 of SEQ ID NO: 7 (or an amino acid sequence having at least 90%, 92%, 94%, 96% 98% or 100% sequence identity to SEQ ID NO: 7).

In one embodiment, the extended LH_{N} BoNT/E peptide may extend into the BoNT/E H_{C} portion from amino acid 845 of SEQ ID NO: 8 up to amino acid residue 854, 864, 874, 884, 894, 904, 914 or 924 of SEQ ID NO: 8 (or an amino acid sequence having at least 90%, 92%, 94%, 96%, 98% or 100% sequence identity to SEQ ID NO: 8).

In one embodiment, the extended LH_{N} BoNT/E peptide extends from amino acid residue 1 of SEQ ID NO: 8 up to amino acid residue 854, 864, 874, 884, 894, 904, 914 or 924 of SEQ ID NO: 8 (or an amino acid sequence having at least 90%, 92%, 94%, 96%, 98% or 100% sequence identity to SEQ ID NO: 8).

In one embodiment, the extended LH_{N} BoNT/E peptide lacks (ie does not comprise) the last 50 C-terminal amino acids of BoNT/E holotoxin. In another embodiment, the extended LH_{N} BoNT/E peptide lacks the last 100, 150, 200, 250, or 300 C-terminal amino acid residues of a BoNT/E holotoxin.

Thus, in one embodiment, the extended LH_{N} BoNT/E peptide lacks (ie. does not comprise) the sequence of amino acid residues from amino acid residue 107, 157, 207, 257, 307 or 357 of SEQ ID NO: 7 to amino acid residue 407 of SEQ ID NO: 7 (or an amino acid sequence having at least 90%, 92%, 94%, 96%, 98% or 100% sequence identity to SEQ ID NO: 7).

In one embodiment, the extended LH_{N} BoNT/E peptide lacks (ie. does not comprise) the sequence of amino acid residues from amino acid residue 951, 1001, 1051, 1101, 1151 or 1201 of SEQ ID NO: 8 to amino acid residue 1251 of SEQ ID NO: 8 (or an amino acid sequence having at least 90%, 92%, 94%, 96%, 98% or 100% sequence identity to SEQ ID NO: 8).

For example, an extended LH_{N} fragment of a BoNT/E may comprise (or consist of) an amino acid sequence having at least 90% identity to SEQ ID NO: 9, such as at least 92%, 94%, 96%, 98% or 100% identity to SEQ ID NO: 9.

In one embodiment, the extended LH_{N} BoNT/E peptide may extend into the BoNT/E H_{C} portion from amino acid 845 of SEQ ID NO: 9 up to amino acid residue 854, 864, 874, 884, 894, 904, 914 or 924 of SEQ ID NO: 9 (or an amino acid sequence having at least 90%, 92%, 94%, 96%, 98% or 100% sequence identity to SEQ ID NO: 9).

In one embodiment, the extended LH_{N} BoNT/E peptide extends from amino acid residue 1 of SEQ ID NO: 9 up to amino acid residue 854, 864, 874, 884, 894, 904, 914 or 924 of SEQ ID NO: 9 (or an amino acid sequence having at least 90%, 92%, 94%, 96%, 98% or 100% sequence identity to SEQ ID NO: 9).

As mentioned above, the BoNT/E peptide of the present invention lacks a functional H_{C} portion—in other words, in contrast to BoNT/E holotoxin, the BoNT/E peptide of the invention does not bind to the presynaptic muscular junction.

Thus, in one embodiment, the BoNT/E peptide of the invention has diminished (or abolished) ability to bind to receptors that are the natural cell surface receptors to which native BoNT/E holotoxin binds. In one embodiment, in contrast to a naturally-occurring BoNT/E holotoxin, the BoNT/E peptide of the invention has a reduced (or abolished) ability to bind to the presynaptic muscular junction.

Any one of a number of routine tests are available to determine the binding ability of a peptide to the natural cell surface receptors to which native BoNT/E binds. By way of example, one conventional test for binding activity, based on binding to rat synaptosomal membranes, is described in Shone et al. (1985) Eur. J. Biochem. 151, 75-82 (incorporated herein by reference). An alternative assay is to measure binding of the fusion protein to immobilised gangliosides such as GT1b, as described in Sinha et al., (2000) Molecular Microbiol. 37, 1041-1051 (incorporated herein by reference).

In one embodiment, the ability of the BoNT/E peptide of the invention to bind to the natural cell surface receptors to which native BoNT/E holotoxin binds is reduced by at least 75%, or by at least 80, 90, 95, 99 or 100% as compared with naturally occurring BoNT/E heavy chain H_{C} portion. In one embodiment, the BoNT/E peptide of the invention exhibits less than 25% of the binding ability of naturally occurring BoNT/E heavy chain H_{C} portion, such as less than 20, 10, 5 or 1% of the binding ability.

Lack of H_{C} binding functionality may be readily conferred by simple omission of part or all of the H_{C} portion. Thus, in one embodiment, the BoNT/E peptide lacks a H_{C} portion of a clostridial neurotoxin heavy chain.

Alternatively, the BoNT/E peptide may comprise part or all of the H_{C} portion, wherein the lack of H_{C} binding function may be conferred by mutation and/or deletion of particular H_{C} amino acid residues present in the H_{C} portion of BoNT/E.

Thus, in one embodiment, the BoNT/E peptide of the invention is modified (as compared with the corresponding amino acid sequence of naturally occurring botulinum neurotoxin) to abolish (or greatly reduce) toxicity. In one embodiment, the BoNT/E peptide of the invention comprises one or more amino acid mutations, selected from amino acid deletions, insertions or substitutions (as compared with the amino acid sequence of naturally occurring BoNT/E), that abolish (or greatly reduce) toxicity.

Mutation(s) to (at least partially) inactivate the binding activity of a BoNT/E peptide as compared with BoNT/E heavy chain H_{C} portion may be selected based on amino acid sequence alignment of the BoNT/E peptide with a corresponding H_{C} sequence from a BoNT/E polypeptide known to have reduced (or abolished) native binding activity.

For example, for BoNT/E, modification of the ASTWYY sequence in the H_{C} domain (eg. to ASTLFY or ASTLYY or ASTWFY)—for example by way of example, substitution of W1224A or W1224L and/or Y1225A or Y1225F (BoNT/E holotoxin) will effectively eliminate the binding activity of the H_{C} domain. This, or a similar mutagenesis strategy, is an option for a type E vaccine which contains part of or even the entire H_{C} domain.

One embodiment of the invention provides a soluble, non-toxic fragment derived from BoNT/E, which provides a protective immune response in a mammal, preferably man, against BoNT/E. In a preferred embodiment, said protective immune response is observed across BoNTE₃ in combination with one or more of BoNT/E₁ and/or BoNT/E₂.

In another embodiment of the invention, a vaccine/antigenic composition for BoNT/E is provided, which contains the light chain and translocation domains of a BoNT/E, preferably type E₃, or fragments or derivatives of these domains.

In another aspect of the invention, a vaccine/antigenic composition for BoNT/E toxin is provided, which is derived from the sequence of botulinum type E neurotoxin subtype E₃.

In another aspect of the invention a vaccine/antigenic composition for BoNT/E toxin is provided which is (based on) the LH_{N} fragment (or a fragment thereof) of BoNT/E neurotoxin subtype E₃.

In one embodiment the BoNT/E peptide comprises any four amino acid residues selected from H142, G176, S198, I199, T230, C231, I232, Q236, R244, K245, I247, N258, V265, Y268, N277, R280, Q295, I302, Q304, E305, D325, L328, A340, E349, K397, and L773.

In another embodiment the BoNT/E peptide comprises any eight amino acid residues selected from H142, G176, S198, I199, T230, C231, I232, Q236, R244, K245, I247, N258, V265, Y268, N277, R280, Q295, I302, Q304, E305, D325, L328, A340, E349, K397, and L773.

In a further embodiment the BoNT/E peptide comprises any twelve amino acid residues selected from H142, G176, S198, I199, T230, C231, I232, Q236, R244, K245, I247, N258, V265, Y268, N277, R280, Q295, I302, Q304, E305, D325, L328, A340, E349, K397, and L773.

In another embodiment the BoNT/E peptide comprises any sixteen amino acid residues selected from H142, G176, S198, I199, T230, C231, I232, Q236, R244, K245, I247, N258, V265, Y268, N277, R280, Q295, I302, Q304, E305, D325, L328, A340, E349, K397, and L773.

In a further embodiment the BoNT/E peptide comprises any twenty amino acid residues selected from H142, G176, S198, I199, T230, C231, I232, Q236, R244, K245, I247, N258, V265, Y268, N277, R280, Q295, I302, Q304, E305, D325, L328, A340, E349, K397, and L773.

In a preferred embodiment the BoNT/E peptide comprises H142, G176, S198, I199, T230, C231, I232, Q236, R244, K245, I247, N258, V265, Y268, N277, R280, Q295, I302, Q304, E305, D325, L328, A340, E349, K397, and L773.

The preferred amino acid residues are selected from one or more of:

### (i) H142, G176, S198, and I199;

### (ii) T230, C231, I232, and Q236;

(iii) 0236, R244, K245, and 1247;

### (iv) N258, V265, Y268, and N277;

### (v) R280, Q295, I302, and Q304;

(vi) E305, D325, L328, and A340; and/or
(vii) E349, K397, and L773.

In one embodiment of the invention, a vaccine/antigenic composition is provided (based on) a peptide consisting of amino acid residues 2-845 (SEQ ID NO: 1) of BoNT/E, or a fragment thereof.

In one embodiment, the BoNT/E peptide of the invention has reduced endopeptidase activity as compared with naturally-occurring BoNT/E holotoxin. In one embodiment, the BoNT/E peptide is endopeptidase negative, meaning that the BoNT/E peptide has little or substantially no residual enzymatic activity as compared to its natural substrate, a SNARE protein such as SNAP-25, syntaxin or VAMP.

In one embodiment, the BoNT/E peptide of the invention has at least 75% less endopeptidase activity than naturally occurring BoNT/E light chain, such as at least 80, 90, 95, 99 or 100% less endopeptidase activity. In one embodiment, the BoNT/E peptide of the invention exhibits less than 25% of the endopeptidase activity of naturally occurring BoNT/E light chain, such as less than 20, 10, 5 or 1% of the endopeptidase activity. In one embodiment, the BoNT/E peptide of the invention is endopeptidase negative.

The endopeptidase activity of a BoNT/E peptide may be determined as a matter of routine, using conventional assays.

By way of example, Hallis et al. (1996) J. Clinical Microbiol., Vol. 34, pages 1934-1938 (incorporated herein by reference), describes an in vitro cell-free system containing immobilised SNAP-25. Cleavage of SNAP-25 protein is measured by using specific antisera raised to the cleavage product.

Alternatively, BoNT endopeptidase activity (ie. SNARE protein cleavage) can be measured by SDS-PAGE and Western Blotting followed by densitometer analysis of the cleaved products. A lesser observed SNARE protein cleavage for the fusion protein versus that observed for a naturally-occurring BoNT holotoxin of said same serotype in conventional assays confirms that the fusion protein has reduced (or abolished) endopeptidase activity.

The substantial absence of detectable endopeptidase activity in any one of these (or other similar) conventional assays means that the BoNT/E peptide is (substantially) “endopeptidase negative”.

Methods are known in the art for reducing the endopeptidase activity of a botulinum neurotoxin or neurotoxin fragment (e.g., LH_{N}). By way of example, it is known to modify one or two or more amino acids of a BoNT/E or an L-chain fragment thereof to reduce the endopeptidase activity as compared with naturally occurring BoNT/E holotoxin.

Thus, in one embodiment, the BoNT/E peptide may contain one or more amino acid mutations (i.e. one or more deletions, substitutions, and/or insertions) within the light chain domain to render it substantially (eg. completely) non-toxic. In one embodiment, said one or more amino acid mutations render the BoNT/E peptide of the invention endopeptidase negative.

Mutation(s) to (at least partially) inactivate the endopeptidase activity of the BoNT/E peptide of the invention may be selected based on amino acid sequence alignment of the L-chain portion of the BoNT/E peptide with a corresponding L-chain sequence from a known endopeptidase-negative (or endopeptidase diminished) BoNT/E polypeptide.

By way of example, the BoNT/E peptide may contain 1 or 2 mutations selected from Glu 213 to Gln and/or His 216 to Tyr. These mutations are illustrated by reference to the specific peptide sequences of SEQ ID NOs: 2 and 3. Reference to said SEQ ID NOs in this context is purely for illustrative purposes, and the illustrated mutations are not limited to the specific SEQ ID NOs.

Additional or alternative mutation(s) to (at least partially) inactivate the metalloprotease activity of the endopeptidase activity of L-chain component may be selected based on simple amino acid sequence alignment of the BoNT/E peptide of the present invention with the corresponding L-chain sequence from a known endopeptidase-negative (or endopeptidase diminished) clostridial neurotoxin species/serotypes. By way of example, a known metalloprotease-inactivating mutation in BoNT/A is known to comprise a substitution/deletion of Glu262. Thus, by simple sequence alignment, the corresponding BoNT/E amino acid residue Glu250 may be similarly substituted/deleted in a BoNT/E peptide of the present invention.

Yet further metalloprotease-inactivating mutations that are known to confer an endopeptidase negative phenotype, include, but are not limited to modification of the HELM active site motif to a HQLIY (i.e. substitutions at residues E213→Q and/or H216→Y); or modification of the HELIH active site motif to a HALIY or HQLIH motif.

In one embodiment, the BoNT/E peptide of the invention has a common antigenic cross-reactivity to a botulinum neurotoxin of serotype E.

As used herein, ‘common antigenic cross-reactivity’ means that an antibody capable of binding to the BoNT/E peptide of the invention would be also capable of binding to a naturally-occurring botulinum neurotoxin of serotype E.

Alternatively, or in addition, ‘common antigenic cross-reactivity’ means that the BoNT/E peptide of the invention induces a “recall response” of a T-lymphocyte that has previously been exposed to a naturally-occurring botulinum neurotoxin of serotype E.

Accordingly, the BoNT/E peptide of the invention provides a neutralising antibody response to a BoNT/E.

The principal BoNT/E peptide of the invention may be readily employed in a vaccine regimen in combination with one or more non-serotype E BoNT peptides, thereby providing a multivalent vaccine.

The non-serotype E peptide(s) may be included in the same vaccine composition as the BoNT/E peptide. Thus, in one embodiment, a multivalent vaccine composition comprises the BoNT/E peptide of the invention and one or more non-serotype E BoNT peptides. In this regard, the BoNT/E peptide is administered simultaneously with the one or more non-serotype E BoNT peptides.

Alternatively, the non-serotype E BoNT peptide(s) may be employed as separate vaccine composition(s). Thus, in one embodiment, a set of vaccine compositions comprises a vaccine composition comprising the BoNT/E peptide of the invention and one or more separate, independent vaccine compositions each comprising one or more non-serotype E BoNT peptides. In this regard, the non-serotype E peptide(s) may be administered prior to the BoNT/E peptide, simultaneously with the BoNT/E peptide, and/or subsequent to the BoNT/E peptide.

In one embodiment, the non-serotype E peptide(s) are selected from a BoNT/A, BoNT/B and/or BoNT/F peptides.

In this regard, non-serotype E BoNT peptides are BoNT LH_{N} fragments (such as BoNT/A, BoNT/B and/or BoNT/F LH_{N} fragments) that lack a functional H_{C} binding portion. Examples of non-serotype E peptides are an LH_{N} fragment from BoNT/A subtype A1, A2, A3 or A4; an LH_{N} fragment from BoNT/B subtype B1, B2, B3 or B4; and/or an LH_{N} fragment from BoNT/F subtype F1, F2 or F barati.

In one embodiment, the one or more non-serotype E BoNT peptides comprise (or consist of) an amino acid sequence that is at least 90%, 92%, 94%, 96%, 98% or 100% identical to an amino acid sequence selected from the group consisting of SEQ ID NOs: 10, 11, 12 and 13 (BoNT/A1, A2, A3 and A4 LH_{N} peptides); SEQ ID NOs: 14, 15, 16 and 17 (BoNT/B1, B2, B3 and B4 LH_{N} peptides); and SEQ ID NOs: 18, 19 and 20 (BoNT/F1, F2 and F3 barati LH_{N} peptides).

As described above for the BoNT/E peptide(s), the non-serotype E peptide(s) may lack the binding ability of BoNT holotoxin to bind to the neuromuscular junction. In one embodiment, the non-serotype E peptide(s) may lack a H_{C} portion of a clostridial neurotoxin heavy chain.

Alternatively (or in addition), the non-serotype E peptide(s) may include all or art of a BoNT heavy chain H_{C} portion, wherein the reduced (or lack of) H_{C} function is conferred by one or more amino acid modifications (eg. deletion, insertion or substitutions) as compared with naturally occurring BoNT of that serotype. Suitable amino acid modifications to non-serotype E BoNT polypeptides, which are known to reduce or abolish BoNT H_{C} binding function, include, but are not limited to the following mutations in the ganglioside binding pocket of H_{C}:
For BoNT/A, modification of the SNWYNR sequence in the H_{C} domain to SNLFNR or SNLYNR or SNWFNR (ie., for A1, substitution of W1266→L and/or Y1267→F);
For BoNT/B, modification of the CISKWYL sequence in the H_{C} domain to CISKLFL or CISKLYL or CISKWFL (ie., for B1, substitution of W1262→L and/or Y1263→F);
For BoNT/F, modification of the LVASSWYY sequence in the H_{C} domain to LVASSLFY or LVASSLYY or LVASSWFY (ie., for F1, substitution of W1250→L and/or Y1251→F).

Also as described above for the BoNT/E peptide(s), the non-serotype E BoNT peptide(s) may include mutations that reduce or destroy native L-chain endopeptidase activity. Amino acid modifications to non-serotype E BoNT polypeptides that are known to confer an endopeptidase negative phenotype, include, but are not limited to:
for BoNT/A, a modification of the HELIH active site motif to a HQLIY motif (i.e. substitution of residue E224→Q and/or H227→Y); or modification of the HELIH active site motif to a HALIY or HQLIH motif; and/or substitution/deletion of residue Glu262;
for BoNT/B, modification of the HELIH active site motif to a HQLIY motif (i.e. substitution of residue E231→Q and/or H234→Y); or modification of the HELIH active site motif to a HALIY or HQLIH motif;
for BoNT/F, modification of the HELIH active site motif to a HQLIY motif (i.e. substitutions at residues E228→Q and/or H231→Y for F1 and F2, and substitutions at residues E220 →Q and/or H223→Y for F3 (barati)); or modification of the HELIH active site motif to a HALIY or HQLIH motif;

In one embodiment, the non-serotype E BoNT peptide has a common antigenic cross-reactivity to a botulinum neurotoxin of said same serotype. Thus, in one embodiment, the BoNT/A peptide has a common antigenic cross-reactivity to a botulinum neurotoxin of serotype A. In one embodiment, the BoNT/B peptide has a common antigenic cross-reactivity to a botulinum neurotoxin of serotype B. In one embodiment, the BoNT/F peptide has a common antigenic cross-reactivity to a botulinum neurotoxin of serotype F.

As used herein, ‘common antigenic cross-reactivity’ means that an antibody capable of binding to the BoNT peptide would be also capable of binding to a naturally-occurring botulinum neurotoxin of the same serotype. Thus, for example, an antibody capable of binding to the BoNT/A peptide would be also capable of binding to a naturally-occurring botulinum neurotoxin of the serotype A. Thus, for example, an antibody capable of binding to the BoNT/B peptide would be also capable of binding to a naturally-occurring botulinum neurotoxin of the serotype B. Thus, for example, an antibody capable of binding to the BoNT/F peptide would be also capable of binding to a naturally-occurring botulinum neurotoxin of the serotype F.

Alternatively, or in addition, ‘common antigenic cross-reactivity’ means that the BoNT peptide induces a “recall response” of a T-lymphocyte that has previously been exposed to a naturally-occurring botulinum neurotoxin of the same serotype. Thus, for example, the BoNT/A peptide induces a “recall response” of a T-lymphocyte that has previously been exposed to a naturally-occurring botulinum neurotoxin of serotype A. Thus, for example, the BoNT/B peptide induces a “recall response” of a T-lymphocyte that has previously been exposed to a naturally-occurring botulinum neurotoxin of serotype B. Thus, for example, the BoNT/F peptide induces a “recall response” of a T-lymphocyte that has previously been exposed to a naturally-occurring botulinum neurotoxin of serotype F.

Accordingly, the BoNT/A peptide provides a neutralising antibody response to a BoNT/A, the BoNT/B peptide provides a neutralising antibody response to a BoNT/B, and the BoNT/F peptide provides a neutralising antibody response to a BoNT/F.

In one embodiment, a BoNT/E peptide of the invention is used in a vaccine regimen in combination with one or more peptides selected from BoNT/A peptides and BoNT/B peptides (as defined above).

For example, a BoNT/E peptide (of subtype E₁, E₂ or E₃) may be used in a vaccine regimen with one or more of a BoNT/A peptide and a BoNT/B peptide. As discussed above, BoNT/A and BoNT/B peptides may lack a functional H_{C} binding portion and/or may include mutations that reduce or destroy native L-chain endopeptidase activity. For example, a BoNT/E peptide of the invention may be used in a vaccine regimen with one or more BoNT/A LH_{N} fragment peptides (such as a BoNT/A LH_{N} fragment peptide of subtype A1, A2, A3 or A4) and/or one or more BoNT/B LH_{N} fragment peptides (such as a BoNT/B LH_{N} fragment peptide of subtype B1, B2, B3 or B4).

In one embodiment, a BoNT/E LH_{N} fragment of subtype E3 is used in a vaccine regimen with a BoNT/A LH_{N} fragment of subtype A1 and/or a BoNT/B LH_{N} fragment of subtype B1, thereby providing a multivalent vaccine that provides protection against BoNT serotypes A, B and E. For example, a trivalent vaccine regimen or composition may comprise a BoNT/E LH_{N} fragment of subtype E3, a BoNT/A LH_{N} fragment of subtype A1 and a BoNT/B LH_{N} fragment of subtype B1.

In one embodiment, a BoNT/E LH_{N} fragment of subtype E3 is used in a vaccine regimen with a BoNT/A LH_{N} fragment comprising (or consisting of) an amino acid sequence having at least 90%, 92%, 94%, 96%, 98% or 100% identical to an amino acid sequence of SEQ ID NO: 10 and/or a BoNT/B LH_{N} fragment comprising (or consisting of) an amino acid sequence having at least 90%, 92%, 94%, 96%, 98% or 100% identical to an amino acid sequence of SEQ ID NO: 14.

The BoNT/A1 and/or BoNT/B1 peptide(s) may be included in the same vaccine composition as the BoNT/E3 peptide. In this regard, the BoNT/E3 peptide is administered simultaneously with the BoNT A1 and/or BoNT/B1 peptides.

Alternatively, the BoNT/E3, BoNT/A1 and/or BoNT/B1 peptide(s) may be employed as separate, independent vaccine composition(s). Thus, in one embodiment, a set of vaccine compositions comprises a vaccine composition comprising the BoNT/E3 peptide of the invention and one or more separate, independent vaccine compositions each comprising a BoNT/A1 peptide and/or a BoNT/B1 peptide. In this regard, the BoNT/A1 and/or BoNT/B1 peptide(s) may be administered prior to the BoNT/E peptide, simultaneously with the BoNT/E peptide, and/or subsequent to the BoNT/E peptide.

In a preferred embodiment, the BoNT peptide(s) may be treated with a chemical modifying agent, which induces the formation of one or more intra-molecular (eg. methylene) bonds. Since the intra-molecular bond(s) are introduced by a modifying chemical, said bonds are typically absent from the corresponding native (ie. untreated) peptide. Two, three or more such bonds may be formed. In this regard, the bond(s) may stem from arginine and/or lysine amino acid residues. A variety of chemical modifying agents may be employed so long as the agent introduces at least one intramolecular bond (e.g. a methylene bond) into the peptide. Examples of suitable cross-linking compounds are given in Table 3. In the case of BoNT/E peptides having two peptide chains (e.g. a L-chain and a H-chain), the intra-molecular bond(s) may form within either chain, and/or across the chains. Said bond(s) may bridge both chains—e.g. the L-chain and H-chain may be bridged by intra-molecular bond(s).

Treatment with a modifying agent in accordance with the present invention is designed to give minimal aggregation of the peptide vaccine and is carried out over a relatively brief incubation period compared with the traditional toxoiding process used in first generation vaccine botulinum candidates, which required an incubation period of up to 25 days.

Thus, in one embodiment of the invention a vaccine is based on a BoNT peptide, which is treated with a modifying agent (eg. formaldehyde) at a relatively low concentration of modifying agent for a relatively brief incubation period. In more detail, a typical incubation period may be up to about 72 hours, eg. up to about 48 hours, or up to about 36 hours, or up to about 24 hours. The incubation temperature is typically up to about 45° C., such as up to about 40° C., or up to about 35° C. In this regard, the minimum incubation temperature is typically higher than about 15° C., such as higher than about 20° C., or higher than about 25° C., or higher than about 30° C. By way of example, incubation temperature ranges may include 30-37° C., 20-24° C. and 3-7° C. For formaldehyde, the modifying agent may be employed typically at a concentration of up to about 2%, such as up to about 1%, eg. up to about 0.75%, or up to about 0.5%, or up to about 0.25% (v/v or w/w based on the ratio of modifying agent to the total reaction mix). In one embodiment, the concentration range for modifying agent is between about 0.1 and 0.3%, eg. between about 0.15 and 0.25%, or about 0.2% (v/v or w/w based on the ratio of modifying agent to the total reaction mix). For other cross-linking agents it may be preferable to define the ratio of cross-linking agent to protein as a molar ratio. The modifying agent may be employed typically at a concentration of cross-linker: peptide molar ratio of 50:1, or up to about 25:1, or up to about 20:1, or up to about 15:1, or up to about 10:1. In one embodiment, a molar ratio for modifying agent: protein is between about 3:1 and 50:1, such as between about 10:1 and 25:1. Studies have established that treatment of peptide preparations treated with 0.2% formaldehyde at 35° C. for 24 hours resulted in intra-molecular cross-linking.

According to one embodiment, the BoNT/E peptide of the invention protects against a challenge of 1000 mouse lethal doses of botulinum type E toxin after a single vaccination. In this regard, it is particularly surprising that a BoNT/E peptide of the present invention is soluble and/or highly efficacious. In contrast, a similar vaccine (based on the sequence of a BoNT/E₁ neurotoxin, SEQ ID NO: 6) is insoluble and has no measurable efficacy.

In one embodiment, the BoNT/E peptide of the present invention provides particularly good protection against a BoNT/E₃, but also provides sound protection across all type E toxin serotypes (e.g. E₁, E₂, and E₃).

Thus, according to a related aspect of the present invention, there is provided use of the above described BoNT/E peptide for stimulating an immune response in an animal, such as in a human. The present invention also provides use of said BoNT/E peptide for protecting against BoNT poisoning, eg. against BoNT/E poisoning.

A related aspect includes a method for stimulating an immune response in an animal, comprising administering an effective amount of the above-described BoNT/E peptide to an animal, such as in a human. The present invention also provides a method for protecting against BoNT poisoning, eg. against BoNT/E poisoning.

A further related aspect includes the above-described BoNT/E peptide, for use in stimulating an immune response in an animal, such as in a human. The present invention also provides said BoNT/E peptide for use in protecting against BoNT poisoning, eg. against BoNT/E poisoning.

The antigenic composition/vaccine of the present invention (hereinafter, simply referred to as vaccine for convenience) may be administered by conventional routes eg. intravenous, subcutaneous, intraperitoneal, and mucosal routes.

Typically, such vaccines are prepared as injectables, either as liquid solutions or suspensions; solid forms suitable for solution in, or suspension in, liquid prior to injection may also be prepared. The preparation may also be emulsified, or the peptide encapsulated in liposomes or microcapsules.

The active immunogenic ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include but are not limited to: aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1-2″-di palmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. Examples of buffering agents include, but are not limited to, sodium succinate (pH 6.5), and phosphate buffered saline (PBS; pH 6.5 and 7.5).

The vaccines are conventionally administered parenterally, by injection, for example, either subcutaneously or intramuscularly.

The vaccines are administered in a manner compatible with the dosage formulation, and in such amount as will be prophylactically and/or therapeutically effective. The quantity to be administered, which is generally in the range of 5 micrograms to 250 micrograms of antigen per dose, depends on the subject to be treated, capacity of the subject's immune system to synthesize antibodies, and the degree of protection desired. Precise amounts of active ingredient required to be administered may depend on the judgment of the practitioner and may be particular to each subject.

The vaccine may be given in a single dose schedule, or optionally in a multiple dose schedule. A multiple dose schedule is one in which a primary course of vaccination may be with 1-6 separate doses, followed by other doses given at subsequent time intervals required to maintain and or reinforce the immune response, for example, at 1-4 months for a second dose, and if needed, a subsequent dose(s) after several months. The dosage regimen will also, at least in part, be determined by the need of the individual and be dependent upon the judgment of the practitioner.

In addition, the vaccine containing the immunogenic antigen(s) may be administered in conjunction with other immunoregulatory agents, for example, immunoglobulins, as well as antibiotics.

Additional formulations which are suitable for other modes of administration include microcapsules, suppositories and, in some cases, oral formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10%-95% of active ingredient, preferably 25%-70%.

A further aspect of the present invention provides a nucleic acid (eg. a DNA) that encodes the above described BoNT/E peptide. The nucleic acid may take the form of a vector, optionally including a promoter and/or terminator. One example of a vector is a plasmid, which optionally includes an origin of replication. Said nucleic acid may be administered to an animal in the form of a nucleic acid vaccine—thus, the nucleic acid aspect may be used to elicit an immune response against BoNT (such as BoNT/E) and/or to provide protection against BoNT poisoning (such as BoNT/E poisoning).

A further aspect comprises expression of the above nucleic acid, vector or plasmid in a host cell (e.g. E. coli). The translated BoNT/E peptide may be recovered by conventional purification protocols.

### DEFINITIONS

BoNT: botulinum neurotoxin.
BoNT/E: botulinum neurotoxin type E
LH_{N}: a fragment of a clostridial neurotoxin (botulinum or tetanus) of approximately 100 kDa which may be a single-chain or di-chain molecule comprising the light chain and the H_{N} domain. The latter domain represents the N-terminal 50 kDa of the neurotoxin heavy chain and is closely associated with light chain domain in the fragment.
Sequence homology: the present invention provides polypeptides that are substantially homologous to a polypeptide based on any one of the SEQ ID NOs set forth herein (including fragments thereof). The term “substantially homologous” is used herein to denote polypeptides having at least 90%, 91%, 92%, 93%, 94%, 95%, 96%, 97%, 98%, 99% or more, sequence identity to the other polypeptide. Exemplary peptides recited in the present application are illustrated by reference to SEQ ID NOs.

Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 1 (amino acids are indicated by the standard one-letter codes). The percent identity is then calculated as:

$$\frac{Total number of identical matches}{[ \begin{matrix}\begin{matrix}length of the longer sequence plus the \\ number of gaps introduced into the longer \\ sequence in order to align\end{matrix} \\ the two sequences\end{matrix} ]} \times 100$$

Substantially homologous polypeptides are characterized as having one or more amino acid substitutions, deletions or additions. These changes are preferably of a minor nature, that is conservative amino acid substitutions (see Table 2) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the clostridial polypeptides of the present invention. A limited number of non-conservative amino acids, amino acids that are not encoded by the genetic code, and unnatural amino acids may be substituted for clostridial polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into peptides. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coil S30 extract and commercially available enzymes and other reagents. Peptides are purified by chromatography. See, for example, Robertson et al., J. Am. Chem. Soc. 113:2722, 1991; Ellman et al., Methods Enzmmol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for clostridial amino acid residues.

Essential amino acids in the clostridial polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related cystatin family members.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Reference to peptides throughout the present application embraces fragments thereof. In particular, the present invention embraces fragments having at least 200 contiguous amino acid residues of a peptide (incl. substantially homologous embodiments thereof) recited in the present application—exemplary peptides are illustrated by specific amino acid SEQ ID NOs. The fragments may embrace at least 200, 300, 400, 500, 600, 700, or at least 800 contiguous amino acids of a peptide (incl. substantially homologous embodiments thereof) recited in the present application.

By way of example, one fragment of the present invention comprises an amino acid sequence having at least 90%, 92%, 94%, 96%, 98% or 100% sequence identity, over a sequence (eg. starting at position 150 or 200) of at least 400 contiguous amino acid residues of SEQ ID NO: 1. Another preferred fragment comprises an amino acid sequence having at least 90%, %, 92%, 94%, 96%, 98% or 100% sequence identity, over a sequence (preferably staring at position 125 or 175) of at least 400 contiguous amino acid residues of SEQ ID NO: 1. A further preferred fragment comprises an amino acid sequence having at least 90%, %, 92%, 94%, 96%, 98% or 100% sequence identity, over the contiguous residues 275-575 of the amino acid sequence of SEQ ID NO: 1.

Antibodies raised against fragments preferably have the property of recognising the full-length counterpart peptide from which they are derived. For example, an antibody raised against an LH_{N}/E fragment of the present invention will preferably have common antigenic cross-reactivity with LH_{N}/E holotoxin.

The BoNT peptides employed in the present invention (eg. the BoNT/E peptide of the invention) lack a functional H_{C} region, and thus have reduced or abolished ability to bind to cell surface receptors that are the natural cell surface receptors to which native botulinum neurotoxin binds. Accordingly, said peptides are not able to bind rat synaptosomal membranes in binding assays as described in Shone et al. (1985) Eur. J. Biochem. 151, 75-82 (incorporated herein by reference).

In one embodiment, the ability of the BoNT peptide to bind to the natural cell surface receptors to which native BoNT holotoxin binds is reduced by at least 75%, or by at least 80, 90, 95, 99 or 100% as compared with naturally occurring BoNT heavy chain H_{C} portion. In one embodiment, the BoNT peptide of the invention exhibits less than 25% of the binding ability of naturally occurring BoNT heavy chain H_{C} portion, such as less than 20, 10, 5 or 1% of the binding ability.

In one embodiment, lack of H_{C} binding functionality may be conferred by omission of the entire H_{C} portion of the heavy chain. Thus, in one example, the BoNT peptide of the invention does not comprise (ie. lacks) a BoNT H_{C} domain.

Alternatively, the BoNT peptide of the invention comprises an extended LH_{N} fragment, though still lacking a functional H_{C} binding domain of BoNT. By way of example, the peptide may be extended (C-terminally) into the H_{C} portion of BoNT. In accordance with this embodiment, the peptide may extend into the BoNT H_{C} portion by any number of consecutive amino acids, so long as the peptide lacks H_{C} binding function (or has reduced H_{C} binding function).

For example, the BoNT peptide of the present invention may include (starting from the N-terminus of the H_{C}) the first 10, 20, 30, 40, 50, 60, 70 or 80 amino acids of a BoNT H_{C} portion.

In one embodiment, the peptide comprises at least 25, 50, 100, 200, 250, 300, 350 or 400 consecutive amino acids of a BoNT heavy chain H_{C} portion. In one embodiment, the peptide comprises less than about 50, 100, 200, 250, 300, 350, 400 or 425 consecutive amino acids of a BoNT heavy chain H_{C} portion.

In one embodiment, the BoNT peptides lack the last 50 C-terminal amino acids of a clostridial neurotoxin holotoxin. In another embodiment, the clostridial peptides lack the last 100, or the last 150, 200, 250 or 300 C-terminal amino acid residues of a clostridial neurotoxin holotoxin.

In one embodiment, the BoNT peptides employed in the invention (eg. the BoNT/E peptide of the invention) may comprise all or part of a BoNT H_{C} domain, wherein the H_{C} binding function is negated/reduced by mutagenesis.

Accordingly, in one embodiment, the BoNT peptide comprises one or more amino acid mutations (selected from one or more amino acid deletions, substitutions or insertions), which reduces (or abolishes) the ability of the BoNT peptide to bind to cell surface receptors that are the natural cell surface receptors to which native BoNT holotoxin binds, as compared with a naturally occurring BoNT heavy chain H_{C} portion.

Mutation(s) to (at least partially) inactivate the binding activity of a BoNT peptide as compared with BoNT heavy chain H_{C} portion may be selected based on amino acid sequence alignment of the BoNT peptide with a corresponding H_{C} sequence from a BoNT polypeptide known to have reduced (or abolished) native binding activity.

By way of example, H_{C} binding function of the peptide is negated/reduced by omission/deletion of one or more amino acids from the H_{C} portion. In one example, the peptide comprises a fragment of a BoNT heavy chain H_{C} portion, wherein said fragment has reduced (or abolished) ability to bind to the natural cell surface receptors to which native BoNT holotoxin binds.

Details of suitable mutations are described in Rummel et al (2004) (Molecular Microbiol. 51:631-634), which is incorporated herein by reference.

By way of example, as discussed above, modification of the ASTWYY sequence in the H_{C} domain of BoNT/E to ASTLFY or ASTLYY or ASTWFY (i.e, for E1, substitution of W1224→L and/or Y1225→F) is known to reduce or abolish BoNT/E H_{C} binding function.

Suitable amino acid modifications to non-serotype E BoNT polypeptides, which are known to reduce or abolish BoNT H_{C} binding function, include, but are not limited to the following mutations in the ganglioside binding pocket of H_{C}:

For BoNT/A, modification of the SNWYNR sequence in the H_{C} domain to SNLFNR or SNLYNR or SNWFNR (i.e, for A1, substitution of W1266→L and/or Y1267→F);

For BoNT/B, modification of the CISKWYL sequence in the H_{C} domain to CISKLFL or CISKLYL or CISKWFL (i.e, for B1, substitution of W1262→L and/or Y1263→F);

For BoNT/F, modification of the LVASSWYY sequence in the H_{C} domain to LVASSLFY or LVASSLYY or LVASSWFY (i.e, for F1, substitution of W1250→L and/or Y1251→F).

Endopeptidase negative: displays no (significant) endopeptidase activity as measured by assays which specifically measure the endopeptidase activities of the botulinum neurotoxins, for example, as described in detail by Hallis et al. (1996) J. Clinical Microbiol. 34:1934-1938. The absence of detectable endopeptidase activity in conventional assays such as the Hallis et al. assay (above) confirms an “endopeptidase negative” phenotype. The Hallis et al. in vitro cell-free system contains immobilised SNAP-25 protein, and cleavage of SNAP-25 is measured by using specific antisera raised to the cleavage product. Alternatively, BoNT endopeptidase activity (ie. SNARE protein cleavage) can be measured by SDS-PAGE and Western Blotting followed by densitometer analysis of the cleaved products. A lesser observed SNARE protein cleavage for the fusion protein versus that observed for BoNT holotoxin in conventional assays confirms that the fusion protein has reduced (or abolished) endopeptidase activity.

In one embodiment, the BoNT peptide has at least 75% less endopeptidase activity than naturally occurring BoNT light chain, such as at least 80, 90, 95, 99 or 100% less endopeptidase activity. In one embodiment, the BoNT peptide of the invention exhibits less than 25% of the endopeptidase activity of naturally occurring BoNT light chain, such as less than 20, 10, 5 or 1% of the endopeptidase activity. In one embodiment, the BoNT peptide of the invention is endopeptidase negative.

Methods are known in the art for reducing the endopeptidase activity of a botulinum neurotoxin or neurotoxin fragment (e.g., LH_{N}). By way of example, it is known to modify one or two or more amino acids of a BoNT or an L-chain fragment thereof to reduce the endopeptidase activity as compared with naturally occurring BoNT holotoxin.

Accordingly, in one embodiment, the BoNT peptide comprises one or more amino acid mutations (selected from one or more amino acid deletions, substitutions or insertions), which reduces (or abolishes) the endopeptidase activity of the BoNT peptide as compared with a naturally occurring BoNT light chain. In one embodiment, said one or more amino acid mutations render the BoNT peptide of the invention endopeptidase negative.

Mutation(s) to (at least partially) inactivate the endopeptidase activity of the BoNT peptide of the invention may be selected based on amino acid sequence alignment of the L-chain portion of the BoNT peptide with a corresponding L-chain sequence from a known endopeptidase-negative (or endopeptidase diminished) BoNT polypeptide.

By way of example, amino acid modifications that are known to confer an endopeptidase negative BoNT/E phenotype, include, but are not limited to modification of the HELIH active site motif to a HQLIY (i.e. substitutions at residues E213→Q and/or H216→Y); or modification of the HELIH active site motif to a HALIY or HQLIH motif; and/or substitution/deletion of residue Glu250.

Amino acid modifications to non-serotype E BoNT polypeptides that are known to confer an endopeptidase negative phenotype, include, but are not limited to:
for BoNT/A, a modification of the HELIH active site motif to a HWY motif (i.e. substitution of residue E224→Q and/or H227→Y); or modification of the HELIH active site motif to a HALIY or HQLIH motif; and/or substitution/deletion of residue Glu262;
for BoNT/B, modification of the HELIH active site motif to a HQLIY motif (i.e. substitution of residue E231→Q and/or H234→Y); or modification of the HELIH active site motif to a HALIY or HQLIH motif;
for BoNT/F, modification of the HELIH active site motif to a HQLIY motif (i.e. substitutions at residues E228→Q and/or H231→Y for F1 and F2, and substitutions at residues E220→Q and/or H223→Y for F3 (barati)); or modification of the HELIH active site motif to a HALIY or HQLIH motif.

Protease cleavage site: native clostridial neurotoxin holotoxin comprises a natural protease cleavage site (eg. a trypsin cleavage site), which is located between the L-chain and the H-chain. Cleavage of this site results in the formation of a di-chain molecule, wherein the L-chain and the H-chain are linked together via a disulphide bond. The polypeptides of the present invention may retain the native cleavage site of holotoxin. Alternatively, they may comprise a non-native cleavage site, which permits ‘controlled’ cleavage of the single chain molecule into its di-chain counterpart. Suitable non-native cleavage sites include

| | |
|---|---|
| Enterokinase | (DDDDK↓) |
| Factor Xa | (IEGR↓/IDGR↓) |
| TEV(Tobacco Etch virus) | (ENLYFQ↓G) |
| Thrombin | (LVPR↓GS) |
| PreScission | (LEVLFQ↓GP). |

Also embraced by the term protease cleavage site is an intein, which is a self-cleaving sequence. The self-splicing reaction is controllable, for example by varying the concentration of reducing agent present.

Vaccine efficacy: the ability of a vaccine to protect animals from the lethal effects of toxins. In one context, this is measured by an ED₅₀ value which is the vaccine dose that will protect animals from a pre-defined challenge dose of toxin. In one format of such an assay, animals are injected with varying doses of the vaccine and then at a defined endpoint (e.g. 28 days from the date if immunisation) are challenged with a lethal dose of toxin (e.g. 1000 mouse lethal doses 50s). The ED₅₀ value is then calculated as the vaccine dose that protects 50% of the animals against the challenge dose of toxin. ED₅₀ values are commonly expressed as micrograms of peptide; the lower the ED₅₀ value, the higher the efficacy of the vaccine.

### EXAMPLES

### Example 1

### Preparation of a Vaccine for Botulinum Type E Neurotoxin

A gene encoding amino acid residues 1-845 of LH_{N}/E was obtained commercially with codon bias suited to expression in E. coli. The gene also contained the mutations: Glu 224 to Gln and H is 227 to Tyr. SEQ ID 1 show the amino acid sequences of an LH_{N}/E vaccine construct of the present invention. LH_{N}/E was expressed from this gene using standard molecular biology methods (e.g. Sambrook et al. 1989, Molecular Cloning a Laboratory Manual, Second Edition, Cold Spring Harbor Laboratory Press, Cold Spring Harbor, N.Y.) and the resulting soluble expressed fragment purified by a combination of hydrophobic interaction chromatography, ion exchange chromatography and ceramic hydroxyl apatite chromatography.

A similar strategy may be applied to the preparation of other type E vaccine sequences. For examples, genes encoding the following residues taken from LH_{N}/E (SEQ ID 1) may be synthesised with codon bias suited to expression in E. coli:

a gene encoding residues 10-845,
a gene encoding residues 50-845,
a gene encoding residues 100-845,
a gene encoding residues 1-835,
a gene encoding residues 1-795,
a gene encoding residues 1-750

Each of these genes may be expressed using standard molecular biology methods (e.g. Sambrook et al. 1989, Molecular Cloning a Laboratory Manual, Second Edition, Cold Spring Harbor Laboratory Press, Cold Spring Harbor, N.Y.) and the resulting expressed fragment purified by a combination of hydrophobic interaction chromatography, ion exchange chromatography and ceramic hydroxyl apatite chromatography.

### Example 2

### Preparation of Further Botulinum Type E Vaccines

Amino acid sequences for a range of additional vaccine LH_{N}/E peptides of the present invention are shown in SEQ IDs 2, 3, 4 and 5. The illustrated peptides form the basis of vaccines of the present invention for botulinum neurotoxin serotype E. These peptides contain mutations to the light chain endopeptidase active site. In these examples, the motif HELIH has been changed to either HQLIH or HQLIY to ablate the enzymic activity. Other mutations to the active site region could also be used to achieve the same ablation of light chain activity, e.g. Glu250. Details of this and other mutations are described in Montecucco et al (2001) (Biochem Biophys Res Comm. 288:1231-7), which is hereby incorporated by reference thereto.

Genes encoding peptides such as the above are commercially available with codon bias for any desired expression host (e.g. E. coli, Pichia pastoris). Peptides are expressed from these genes using standard molecular biology methods (e.g. Sambrook et al. 1989, Molecular Cloning a Laboratory Manual, Second Edition, Cold Spring Harbor Laboratory Press, Cold Spring Harbor, N.Y.) and the resulting soluble expressed peptide is purified by a combination of hydrophobic interaction chromatography, ion exchange chromatography and ceramic hydroxyl apatite chromatography. Other chromatographic techniques well known to the art of protein purification, such size exclusion chromatography and/or affinity chromatography, may be used.

The purified peptide is then dialysed against buffer (10 mm Hepes buffer pH 7.4 containing 100 mM NaCl) and then either stored at −80° C. or formulated as a vaccine

### Example 3

### Formulation and Adsorption to Aluminium Adiuvant

The LH_{N}/E molecules and derivatives were adsorbed to the aluminium hydroxide adjuvant (Alhydrogel™). Preliminary work in optimizing adjuvant binding was performed by examining (a) buffer type, (b) buffer pH, (c) salt concentration and (d) ratio of adjuvant to peptide.

The LH_{N}/E vaccines were formulated by adsorption onto aluminium hydroxide (Alhydrogel®). Formulation conditions were employed such that there may up to 500 μg LH_{N}/A adsorbed per ml of Alhydrogel solution. The following formulation buffer was adopted:

### 10 mM Hepes pH 7.4

### 100 mM NaCl

3100 μg ml Al (approx 0.65-0.95% Alhydrogel depending on batch)

Peptide up to a concentration of 500 μml⁻¹ was added (0.2 ml dose to give up to 100 μg). The 0.2 ml vaccine dose contained 620 μg ml⁻¹ Al. The formulated vaccine was then gently mixed for 6 hours at room temperature and stored at 4° C. until use.

Data showed that LH_{N} peptides can be completely adsorbed to Alhydrogel™ in 10 mM Hepes buffer pH 7.4 containing 100 mM NaCl, to a final concentration of 0.6 mg of aluminium per 0.5 mL dose

### Example 4

### Assessment of Type E Vaccine Efficacy in Monovalent Formulations

Samples of LH_{N}/E vaccine adsorbed onto an adjuvant such Alhydrogel™ were diluted with buffer containing the same adjuvant to give a range of concentrations of antigen. For example, the following vaccine doses in 0.2 ml were used:

| | | | | | | | |
|---|---|---|---|---|---|---|---|
| 20 | 6.67 | 2.22 | 0.74 | 0.24 | 0.08 | 0.027 | 0 μg per 0.2 ml dose |

Doses of the vaccine were then injected into mice (10 mice per vaccine dose; 0.2 ml into each mouse by the sub-cutaneous route). At 28 days post administration, the mice were challenged with a lethal concentration of BoNT/E₁ toxin (1000 LD₅₀ administered into the peritoneal cavity) and any deaths were recorded over a 4 day period post-challenge.

The raw data are shown in Table 4

Calculated ED₅₀ value for the LH_{N}/E construct=0.9±0.09 μg

These data show that the LH_{N}/E constructs have excellent protective efficacy after a single vaccine dose to a challenge of 1000 LD₅₀ BoNT/E.

### Example 5

### Assessment of Vaccine Efficacy in Trivalent A, B and E Formulation

Samples of LH_{N}/E vaccine were adsorbed onto an Alhydrogel™ adjuvant as described in Example 3 in conjunction with LH_{N}/A and LH_{N}/B protein antigens such that each antigen was at a final concentration of 100 μg protein per ml in the formulated vaccine.

The formulated vaccine was diluted with buffer containing the same adjuvant to give a range of concentrations of antigen. For example, the following vaccine doses in 0.2 ml were used:

| | | | | | | | |
|---|---|---|---|---|---|---|---|
| 20 | 6.67 | 2.22 | 0.74 | 0.24 | 0.08 | 0.027 | 0 μg per 0.2 ml dose |

Doses of the vaccine were then injected into mice (10 mice per vaccine dose; 0.2 ml into each mouse by the sub-cutaneous route). Three such experimental groups were injected. At 28 days post administration, the experimental groups mice were challenged with lethal concentrations of either BoNT/A₁, BoNT/B₁ or BoNT/E₁ toxin (1000 LD₅₀ administered was into the peritoneal cavity) and any deaths were recorded over a 4 day period post-challenge.

The raw data are shown in Table 5

Calculated ED₅₀ values for trivalent study:

| | |
|---|---|
| A Challenge | 0.054 ± 0.006 μg |
| B Challenge | 0.079 ± 0.02 μg |
| E Challenge | 0.9 ± 0.09 μg |

These data show that all three LH_{N} constructs have excellent protective efficacy after a single vaccine dose to a challenge of 1000 LD₅₀ of the respective BoNT. In addition, no evidence of immunosuppression in the trivalent formulation. LH_{N}/E ED₅₀ values in the monovalent and trivalent formulations were virtually identical.

### Example 6

### Assessment of Vaccine Efficacy Against Various Sub-Types of Bont/E

LH_{N}/E is adjusted to 1 mg ml⁻¹ with Hepes/NaCl buffer, treated with HCHO (0.2% for 24 h at 35° C.) and adsorbed onto Alhydrogel (3100 μg ml⁻¹ final concentration) in 10 mM Hepes pH 7.4/100 mM NaCl buffer (peptide concentration 100 μml⁻¹).

After mixing and incubation at 4° C. for 4 hours, the mixture is dialysed against the Hepes buffer to remove the formaldehyde and then diluted with buffer containing Alhydrogel to give the following concentrations of antigen per 0.2 ml:

| | | | | | | | |
|---|---|---|---|---|---|---|---|
| 20 | 6.67 | 2.22 | 0.74 | 0.24 | 0.08 | 0.027 | 0 μg per 0.2 ml dose |

At 28 days post administration, 3 test groups of mice are challenged with either BoNT/E₁, BoNT/E₂ or BoNT/E₃ (1000 LD₅₀ i.p. in 0.5 ml). Deaths are recorded over a 4-day period post-challenge.

Vaccine protection against all three BoNT/E sub-types is confirmed.

### Example 7

### Detection of Fragments of the Invention by Immunoassay

Peptide fragments of the invention bear antigenic determinants which are detectable by immunoassays. One or more antigenic determinants is shared by the LH_{N}/E fragments of the present invention and thus antibodies raised against one LH_{N}/E fragment may also bind corresponding LH_{N} fragments of the invention. Immunoassays to detect the presence of fragments of the invention are conducted as follows.

Fragments of the invention are coated onto microtiter plates at concentration of 5 μg/ml in a suitable buffer such 50 mM Hepes pH 7.4 and allowed to bind at 4° C. overnight. After blocking excess protein binding sites with a blocking agent (e.g. 5% foetal bovine serum in PBS), the plate is washed with PBS containing 0.1% tween 20. Antibodies prepared in animals (e.g. rabbits) to the corresponding LH_{N} fragment are then added to the plate wells. The antibody solution is applied at various dilutions, e.g. 1/1000 to 1/1000,000 dilution of the neat serum and allowed to bind for 1 hour at 37° C. After washing with PBS/Tween20, a commercially available ant-rabbit IgG peroxidise conjugate solution is added at e.g. a 1/1000 dilution and allowed to bind for 1 hour at 37° C. The unbound conjugate is then removed by washing with PBS/tween20 and then suitable peroxidise substrates (e.g. 3,3′,5,5′-Tetramethylbenzidine and hydrogen peroxide) added. For wells coated with fragment of the invention, colour will develop which is significantly above background levels indicating the presence of a peptide fragment containing epitopes common to the LH_{N}/E fragment. The test therefore indicates the presence peptide fragment properties consistent with fragments of the invention.

### Example 8

### Preparation of Fragments of the Invention Based on Clostridial Neurotoxins which Contain a Mutated, Dysfunctional H_{C} Domain

An example of the amino acid sequence of a botulinum vaccine which has a non-functional H_{C} domain is shown in SEQ ID No. 8. This peptide contains mutations to ganglioside binding site of the H_{C} domain. In more detail, referring to SEQ ID No. 8, two amino acid residue mutations (W1224 to L and Y1225 to F) in the ganglioside binding pocket cause the H_{C} region to lose its receptor binding function. Other mutations to the active site achieve the same ablation of H_{C} receptor binding activity, e.g. Y1225 Y to S in botulinum type E. Details of this and other mutations are described in Rummel et al (2004) (Molecular Microbiol. 51:631-634), which is hereby incorporated by reference thereto.

Genes encoding peptides such as the above are commercially available with codon bias for any desired expression host (e.g. E. coli, Pichia pastoris). Peptides are expressed from these gene using standard molecular biology methods (e.g. Sambrook et al. 1989, Molecular Cloning a Laboratory Manual, Second Edition, Cold Spring Harbor Laboratory Press, Cold Spring Harbor, N.Y.) and the resulting soluble expressed peptide is purified by a combination of hydrophobic interaction chromatography, ion exchange chromatography and ceramic hydroxyl apatite chromatography. Other chromatographic techniques well known to the art of protein purification, such size exclusion chromatography, may be used.

The peptide is then dialysed against buffer (10 mm Hepes buffer pH 7.4 containing 100 mM NaCl) and then either stored at −80° C. or formulated as a vaccine

### Example 9

### Assays to Demonstrate that Fragments of the Invention do not Possess a Functional H_{C} Receptor Binding Domain

Clostridia' neurotoxin is labelled with 125-iodine using chloramine-T and its binding to various cells assessed by standard methods such as described in Evans et al. 1986, Eur J. Biochem., 154, 409 or Wadsworth et al. 1990, Biochem. J. 268, 123). In competitive binding experiments, native clostridial neurotoxins competes for receptors present on neuronal cells or brain synaptosomes with the radiolabelled botulinum toxin thus, reducing the binding of the latter. This is measured by a reduction in bound radioactive ligand. All binding experiments are carried out in binding buffers, e.g. 50 mM HEPES pH 7.0, 30 mM NaCl, 0.25% sucrose, 0.25% bovine serum albumin. In a typical binding experiment the radiolabelled clostridial neurotoxin is held at a fixed concentration of between 1-20 nM. Reaction mixtures are prepared by mixing the radiolabelled toxin with various higher concentrations (up to 10 μM) of unlabelled neurotoxin or fragment of the invention. The reaction mixtures are then added to neuronal cells or rat brain synaptosomes and are incubated at 0-3° C. for 2 hr. After this period the neuronal cells of synaptosomes are washed twice with ice-cold binding buffer and the amount of labelled clostridia' neurotoxin bound to cells or synaptosomes assessed by γ-counting. In reaction mixtures which contained native neurotoxin, the peptide competes with ¹²⁵I-labelled botulinum type E neurotoxin for neuronal cell receptors and reduces the binding of the latter. However, when a clostridial peptide of the invention is added to reaction mixture no reduction in binding of the labelled toxin occurs. This demonstrates that clostridial peptides of the invention do not contain a function H_{C} binding domain.

### Example 10

### Preparation of Fragments of the Invention that Contain Intramolecular Cross-Links to Enhance Vaccine Efficacy

Purified type E vaccine peptides at a concentration of between 0.2-2 mg/ml are dialysed against a suitable buffer (e.g. 10 mM Hepes buffer pH 7.4 containing 100 mM NaCl) and then formaldehyde added at a final concentration of between 0.05 and 0.5% and incubated for between 24 and 72 hours at 35° C. After incubation, the formaldehyde is removed from the mixture by dialysis. Conditions for the treatment with formaldehyde may vary between peptides and final conditions may be fine-tuned on the basis of outcome of protective efficacy evaluations.

As well as formaldehyde, other cross-linking agents may be employed to produce vaccines of the invention. Examples of cross-linking agents that may be employed are shown in Table 3. The cross linking agent is dissolved in a suitable solvent (e.g. water, buffer, ethanol or acetone) and then mixed with the vaccine peptide. The cross-linking agent is added to the peptide in a molar excess which may be between 3-50 fold moles of cross-linker per mole of peptide. The peptide is typically present at a concentration of between 0.1-5 mg/ml, and is typically incubated with the cross-linker from 1-24 hr at temperature between 4-37° C. Exact conditions may be determined by those which provide the optimal immune response in animals (e.g. mice, guinea pigs or rabbits) (see Example 4).

### Example 11

### Vaccination by Peptide/Peptide Fragments of the Invention

A vaccine, represented by a peptide/peptide fragment of the invention is prepared by current Good Manufacturing Practice. Using such practices, peptides/peptide fragments of the invention may be bound to an adjuvant of aluminium hydroxide which is commercially available (e.g. Alhydrogel). A typical composition comprises:

A buffer (e.g. Hepes buffer between 5 and 20 mM and pH between 7.0 and 7.5
A salt component to make the vaccine physiologically isotonic (e.g. between 100 and 150 mM NaCl
An adjuvant (e.g. aluminium hydroxide at a final aluminium concentration of between 100 and 700 μg per vaccine dose)
A preservative (e.g. Thiomersal at 0.01% or formaldehyde at 0.01%)

Such vaccine compositions are administered to humans by a variety of different immunisation regimens, e.g.
1. A singe dose (e.g. 20 μg adsorbed fragment of the invention) in 0.5 ml administered sub-cutaneously.
2. Two doses (e.g. of 10 μg adsorbed fragment of the invention) in 0.5 mls administered at 0 and 4 weeks.
3. Three doses (e.g. of 10 μg adsorbed fragment of the invention) in 0.5 mls administered at 0, 2 and 12 weeks.

These vaccination regimens confer levels of protection against exposure to the homologous serotypes of botulinum neurotoxins. An antibody response in humans is measured by standard ELISA assays.

During formulation of the fragment of the invention, other antigens may also be included in the formulation. Such antigens may include different botulinum serotype vaccines or antigens not related to the botulinum toxins.

### Example 12

### Assessment of Vaccine Efficacy in Trivalent A, B and E Formulation

A trivalent LHN/A, B and E vaccine was formulated using 20 μg/ml of formaldehyde treated LH_{N}/A and E and non-formaldehyde treated LH_{N}/B monovalent vaccine. All three monovalent vaccines were adjuvanted to alhydrogel and blended together in a 1:1:1 ratio of monovalent vaccine to form the final LH_{N} ABE trivalent vaccine.

Efficacy studies were performed on the monovalent and trivalent vaccine formulations. For the monovalent mouse efficacy studies, groups of 10 mice were vaccinated with various dilutions off the monovalent vaccines and then challenged 28 days later with 1000 MIPLD₅₀ of either BoNT A, B or E toxin and surviving mice were tabulated 4 days later in each group (Table 6).

For the trivalent efficacy studies, three groups of 80 mice were vaccinated with the trivalent vaccine and then 28 days later were challenged with either BoNT A, B or E toxin. Surviving mice were tabulated and ED₅₀ potency values calculated (Table 7).

From comparisons of the monovalent and trivalent ED₅₀ data it is clear that there is no evidence of immune interference or suppression by any of the LH_{N} fragments used for vaccination.

TABLE 1
Alignment scores for determining sequence identity

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

TABLE 2
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

TABLE 3
Examples of Suitable Cross-linking Reagents

C6-succinimidyl 4-hydrazinonicotinate acetone hydrazone
C6-succinimidyl 4-formylbenzoate
BIS-(Sulfosuccinimidyl) suberate
Disuccinimidyl suberate
Dimethyl suberimidate dihydrochloride
Dimethyl pimelimidate 2 HCl
Dimethyl adipimidate dihydrochloride
Formaldehyde
Succinimidyl 4-hydrazidoterephthalate hydrochloride
Disuccinimidyl glutarate

TABLE 4
Single Dose Monovalent LH_{N}/E Efficacy Studies

LH_{N}/E was absorbed onto Alhydrogel (3100 μg ml⁻¹ final
concentration) in 10 mM Hepes pH 7.4/100 mM NaCl buffer
(protein concentration 300 μg ml⁻¹). The antigen was
then diluted with buffer containing Alhydrogel to give
the following concentrations of antigen per 0.2 ml:
20 6.67 2.22 0.74 0.24 0.08 0.027 0 μg per 0.2 ml dose
At 28 days post administration, mice were challenged with
BoNT/E (498Ref pre-trypsin treated)) at 1000 LD₅₀ in 0.5 ml
administered i.p. Deaths were recorded over a 4 day period
post-challenge.

Monovalent Efficacy - Single Dose Raw Data

| Vaccine | Surviving Mice (of 10) |
|---|---|
| Dose | at 4 days Post Challenge |
| (μg) | BoNT/E Challenge |
| 20 | 8 |
| 6.67 | 10 |
| 2.22 | 8 |
| 0.74 | 4 |
| 0.24 | 1 |
| 0.08 | 0 |
| 0.027 | 0 |
| 0 | 0 |

Calculated ED₅₀ values for monovalent study:
E Challenge 0.9±0.15 μg
   The sub-microgram ED₅₀ value indicates high protective efficacy.

TABLE 5
Single Dose Trivalent Efficacy Studies (A, B, E Challenge)
Details of the Test

LH_{N}/A was adjusted to 1 mg ml⁻¹ with Hepes/NaCl buffer, treated
with HCHO (0.2% for 24 h at 35° C.) and adsorbed onto Alhydrogel
(3100 μg ml⁻¹ final concentration) in 10 mM Hepes pH 7.4/100 mM
NaCl buffer (protein concentration 300 μg ml⁻¹). After mixing and
incubation at 4° C. for 4 hours, the mixture was dialysed against
the Hepes buffer to remove the formaldehyde.
LH_{N}/B and LH_{N}/E were each absorbed onto Alhydrogel (3100 μg ml⁻¹
final concentration) in 10 mM Hepes pH 7.4/100 mM NaCl buffer
(protein concentration 300 μg ml⁻¹ for each).
A 1:1:1 mixture of the above antigens was made and then diluted
with buffer containing Alhydrogel to give the following
concentrations of antigen per 0.2 ml:
20 6.67 2.22 0.74 0.24 0.08 0.027 0 μg per 0.2 ml dose
At 28 days post administration, 3 groups of mice were challenged
with either BoNT/A₁ (HPA Batch May 11, 1998 3502A), BoNT/B
(Batch 498Ref Dec. 2, 1999) or BoNT/E (498Ref pre-trypsin
treated)), each at 1000 LD₅₀ in 0.5 ml administered i.p.
Deaths were recorded over a 4 day period post-challenge.

### Results

The raw data from the test are presented in the table below.

### Trivalent Efficacy—Single Dose Raw Data

| Vaccine | Surviving Mice (of 10) at | | |
|---|---|---|---|
| Dose | 4 days Post Challenge | | |
| (μg) | A | B | E |
| 20 | 10 | 10 | 9 |
| 6.67 | 10 | 10 | 9 |
| 2.22 | 10 | 10 | 8 |
| 0.74 | 9 | 10 | 4 |
| 0.24 | 9 | 7 | 0 |
| 0.08 | 6 | 6 | 0 |
| 0.027 | 3 | 1 | 0 |
| 0 | 0 | 0 | 0 |

Calculated ED₅₀ values for trivalent study:

| | |
|---|---|
| A Challenge | 0.054 ± 0.006 μg |
| B Challenge | 0.079 ± 0.02 μg |
| E Challenge | 0.9 ± 0.09 μg |

The sub-microgram ED₅₀ value indicate high protective efficacy

TABLE 6
Monovalent LH_{N}/A, B and E are highly protective
against toxin challenge in mice.

| Vaccine | Number of Surviving Mice (of 10) at 4 Days Post-challenge | | |
|---|---|---|---|
| Dose | Formaldehyde- | | Formaldehyde- |
| (ug protein) | treated LH_{N}/A | LH_{N}/B | treated LH_{N}/E |
| 20 | 10 | 10 | 10 |
| 6.67 | 10 | 10 | 10 |
| 2.22 | 10 | 10 | 10 |
| 0.74 | 10 | 10 | 10 |
| 0.24 | 10 | 10 | 10 |
| 0.08 | 5 | 4 | 8 |
| 0.027 | 3 | 0 | 3 |
| 0 | 0 | 0 | 0 |
| ED₅₀ | 0.056 | 0.083 | 0.041 |
| (ug protein) | | | |

TABLE 7
Trivalent LH_{N} ABE vaccine is highly protective
against toxin challenge in mice.

| Vaccine | Number of Surviving Mice (of 10) at 4 Days Post-challenge | | |
|---|---|---|---|
| Dose | Formaldehyde- | | Formaldehyde- |
| (ug protein) | treated LH_{N}/A | LH_{N}/B | treated LH_{N}/E |
| 20 | 10 | 10 | 10 |
| 6.67 | 10 | 10 | 10 |
| 2.22 | 10 | 10 | 10 |
| 0.74 | 9 | 10 | 10 |
| 0.24 | 10 | 9 | 10 |
| 0.08 | 9 | 7 | 10 |
| 0.027 | 8 | 2 | 7 |
| 0 | 0 | 0 | 0 |
| ED₅₀ | 0.0029 | 0.056 | 0.026 |
| (ug protein) | | | |

### SEQ ID NOs

The following amino acid sequences are described in the present application.

For amino acid sequences that commence with the amino acid residue Methionine (Met), it is understood that this first amino acid residue is optional and can be omitted.

By way of example, the first amino acid residue of SEQ ID NO: 1 as shown below is Met. However, this first Met residue of SEQ ID NO: 1 is optional and can be omitted. If the first amino acid (Met) of SEQ ID NO: 1 is omitted, the revised sequence commences with the 2^{nd} amino acid—ie. Proline (Pro).

SEQ ID NO: 1 BoNT Type E Vaccine (Clostridium botulinum Strain Alaska E43)

MPKINSFNYNDPVNDRTILYIKPGGCQEFYKSFNIMKNIWIIPE
RNVIGTTPQDFHPPTSLKNGDSSYYDPNYLQSDEEKDRFLKIVTKIFNRINNNLSGGI
LLEELSKANPYLGNDNTPDNQFHIGDASAVEIKFSNGSQHILLPNVIIMGAEPDLFET
NSSNISLRNNYMPSNHGFGSIAIVTFSPEYSFRFNDNSINEFIQDPALTLMHELIHSL
HGLYGAKGITTTCIITQQQNPLITNRKGINIEEFLTFGGNDLNIITVAQYNDIYTNLL
NDYRKIASKLSKVQVSNPQLNPYKDIFQEKYGLDKDASGIYSVNINKFDDILKKLYSF
TEFDLATKFQVKCRETYIGQYKYFKLSNLLNDSIYNISEGYNINNLKVNFRGQNANLN
PRIIKPITGRGLVKKIIRFCKNIVSVKGIRKSICIEINNGELFFVASENSYNDDNINT
PKEIDDTVTSNNNYENDLDQVILNFNSESAPGLSDEKLNLTIQNDAYIPKYDSNGTSD
IEQHDVNELNVFFYLDAQKVPEGENNVNLTSSIDTALLEQPKIYTFFSSEFINNVNKP
VQAALFVSWIQQVLVDFTTEANQKSTVDKIADISIVVPYIGLALNIGNEAQKGNFKDA
LELLGAGILLEFEPELLIPTILVFTIKSFLGSSDNKNKVIKAINNALKERDEKWKEVY
SFIVSNWMTKINTQFNKRKEQMYQALQNQVNAIKTIIESKYNSYTLEEKNELTNKYDI
KQIENELNQKVSIAMNNIDRFLTESSISYLMKLINEVKINKLREYDENVKTYLLNYII
QHGSILGESQQELNSMVTDTLNNSIPFKLSSYTDDKILISYFNKFFK

SEQ ID NO: 2 BoNT Type E Vaccine (Clostridium botulinum Strain Alaska E43 Containing a Glu 213 to Gln Mutation)

MPKINSFNYNDPVNDRTILYIKPGGCQEFYKSFNIMKNIWIIPE
RNVIGTTPQDFHPPTSLKNGDSSYYDPNYLQSDEEKDRFLKIVTKIFNRINNNLSGGI
LLEELSKANPYLGNDNTPDNQFHIGDASAVEIKFSNGSQHILLPNVIIMGAEPDLFET
NSSNISLRNNYMPSNHGFGSIAIVTFSPEYSFRFNDNSINEFIQDPALTLMHQLIHSL
HGLYGAKGITTTCIITQQQNPLITNRKGINIEEFLTFGGNDLNIITVAQYNDIYTNLL
NDYRKIASKLSKVQVSNPQLNPYKDIFQEKYGLDKDASGIYSVNINKFDDILKKLYSF
TEFDLATKFQVKCRETYIGQYKYFKLSNLLNDSIYNISEGYNINNLKVNFRGQNANLN
PRIIKPITGRGLVKKIIRFCKNIVSVKGIRKSICIEINNGELFFVASENSYNDDNINT
PKEIDDTVTSNNNYENDLDQVILNFNSESAPGLSDEKLNLTIQNDAYIPKYDSNGTSD
IEQHDVNELNVFFYLDAQKVPEGENNVNLTSSIDTALLEQPKIYTFFSSEFINNVNKP
VQAALFVSWIQQVLVDFTTEANQKSTVDKIADISIVVPYIGLALNIGNEAQKGNFKDA
LELLGAGILLEFEPELLIPTILVFTIKSFLGSSDNKNKVIKAINNALKERDEKWKEVY
SFIVSNWMTKINTQFNKRKEQMYQALQNQVNAIKTIIESKYNSYTLEEKNELTNKYDI
KQIENELNQKVSIAMNNIDRFLTESSISYLMKLINEVKINKLREYDENVKTYLLNYII
QHGSILGESQQELNSMVTDTLNNSIPFKLSSYTDDKILISYFNKFFK

SEQ ID NO: 3 BoNT Type E Vaccine (Clostridium botulinum Strain Alaska E43 Containing Glu 213 to Gln, and His 216 to Tyr Mutations)

MPKINSFNYNDPVNDRTILYIKPGGCQEFYKSFNIMKNIWIIPE
RNVIGTTPQDFHPPTSLKNGDSSYYDPNYLQSDEEKDRFLKIVTKIFNRINNNLSGGI
LLEELSKANPYLGNDNTPDNQFHIGDASAVEIKFSNGSQHILLPNVIIMGAEPDLFET
NSSNISLRNNYMPSNHGFGSIAIVTFSPEYSFRFNDNSINEFIQDPALTLMHQLIYSL
HGLYGAKGITTTCIITQQQNPLITNRKGINIEEFLTFGGNDLNIITVAQYNDIYTNLL
NDYRKIASKLSKVQVSNPQLNPYKDIFQEKYGLDKDASGIYSVNINKFDDILKKLYSF
TEFDLATKFQVKCRETYIGQYKYFKLSNLLNDSIYNISEGYNINNLKVNFRGQNANLN
PRIIKPITGRGLVKKIIRFCKNIVSVKGIRKSICIEINNGELFFVASENSYNDDNINT
PKEIDDTVTSNNNYENDLDQVILNFNSESAPGLSDEKLNLTIQNDAYIPKYDSNGTSD
IEQHDVNELNVFFYLDAQKVPEGENNVNLTSSIDTALLEQPKIYTFFSSEFINNVNKP
VQAALFVSWIQQVLVDFTTEANQKSTVDKIADISIVVPYIGLALNIGNEAQKGNFKDA
LELLGAGILLEFEPELLIPTILVFTIKSFLGSSDNKNKVIKAINNALKERDEKWKEVY
SFIVSNWMTKINTQFNKRKEQMYQALQNQVNAIKTIIESKYNSYTLEEKNELTNKYDI
KQIENELNQKVSIAMNNIDRFLTESSISYLMKLINEVKINKLREYDENVKTYLLNYII
QHGSILGESQQELNSMVTDTLNNSIPFKLSSYTDDKILISYFNKFFK

SEQ ID NO: 4 BoNT Type E Vaccine (Clostridium botulinum Strain Alaska E43 Containing Glu 213 to Gln, and His 216 to Tyr Mutations)

MPVNDRTILYIKPGGCQEFYKSFNIMKNIWIIPE
RNVIGTTPQDFHPPTSLKNGDSSYYDPNYLQSDEEKDRFLKIVTKIFNRINNNLSGGI
LLEELSKANPYLGNDNTPDNQFHIGDASAVEIKFSNGSQHILLPNVIIMGAEPDLFET
NSSNISLRNNYMPSNHGFGSIAIVTFSPEYSFRFNDNSINEFIQDPALTLMHQLIYSL
HGLYGAKGITTTCIITQQQNPLITNRKGINIEEFLTFGGNDLNIITVAQYNDIYTNLL
NDYRKIASKLSKVQVSNPQLNPYKDIFQEKYGLDKDASGIYSVNINKFDDILKKLYSF
TEFDLATKFQVKCRETYIGQYKYFKLSNLLNDSIYNISEGYNINNLKVNFRGQNANLN
PRIIKPITGRGLVKKIIRFCKNIVSVKGIRKSICIEINNGELFFVASENSYNDDNINT
PKEIDDTVTSNNNYENDLDQVILNFNSESAPGLSDEKLNLTIQNDAYIPKYDSNGTSD
IEQHDVNELNVFFYLDAQKVPEGENNVNLTSSIDTALLEQPKIYTFFSSEFINNVNKP
VQAALFVSWIQQVLVDFTTEANQKSTVDKIADISIVVPYIGLALNIGNEAQKGNFKDA
LELLGAGILLEFEPELLIPTILVFTIKSFLGSSDNKNKVIKAINNALKERDEKWKEVY
SFIVSNWMTKINTQFNKRKEQMYQALQNQVNAIKTIIESKYNSYTLEEKNELTNKYDI
KQIENELNQKVSIAMNNIDRFLTESSISYLMKLINEVKINKLREYDENVKTYLLNYII
QHGSILGESQQELNSMVTDTLNNSIPFKLSSYTDDKI

SEQ ID NO: 5 BoNT Type E Vaccine (Clostridium botulinum Strain Alaska E43 Containing Glu 213 to Gln Mutation)

MPVNDRTILYIKPGGCQEFYKSFNIMKNIWIIPE
RNVIGTTPQDFHPPTSLKNGDSSYYDPNYLQSDEEKDRFLKIVTKIFNRINNNLSGGI
LLEELSKANPYLGNDNTPDNQFHIGDASAVEIKFSNGSQHILLPNVIIMGAEPDLFET
NSSNISLRNNYMPSNHGFGSIAIVTFSPEYSFRFNDNSINEFIQDPALTLMHQLIYSL
HGLYGAKGITTTCIITQQQNPLITNRKGINIEEFLTFGGNDLNIITVAQYNDIYTNLL
NDYRKIASKLSKVQVSNPQLNPYKDIFQEKYGLDKDASGIYSVNINKFDDILKKLYSF
TEFDLATKFQVKCRETYIGQYKYFKLSNLLNDSIYNISEGYNINNLKVNFRGQNANLN
PRIIKPITGRGLVKKIIRFCKNIVSVKGIRKSICIEINNGELFFVASENSYNDDNINT
PKEIDDTVTSNNNYENDLDQVILNFNSESAPGLSDEKLNLTIQNDAYIPKYDSNGTSD
IEQHDVNELNVFFYLDAQKVPEGENNVNLTSSIDTALLEQPKIYTFFSSEFINNVNKP
VQAALFVSWIQQVLVDFTTEANQKSTVDKIADISIVVPYIGLALNIGNEAQKGNFKDA
LELLGAGILLEFEPELLIPTILVFTIKSFLGSSDNKNKVIKAINNALKERDEKWKEVY
SFIVSNWMTKINTQFNKRKEQMYQALQNQVNAIKTIIESKYNSYTLEEKNELTNKYDI
KQIENELNQKVSIAMNNIDRFLTESSISYLMKLINEVKINKLREYDENVKTYLLNYII
QHGSILGESQQELNSMVTDTLNNSIPFKLSSYTDDKI

SEQ ID No: 6 BoNT Type E (Lh_{N} from Clostridium botulinum Strain Beluga, which is a Bont/E Subtype E₁ Neurotoxin)

MPKINSFNYNDPVNDRTILYIKPGGCQEFYKSFNIMKNIWIIPERNVIG
TTPQDFHPPTSLKNGDSSYYDPNYLQSDEEKDRFLKIVTKIFNRINNNL
SGGILLEELSKANPYLGNDNTPDNQFHIGDASAVEIKFSNGIQDILLPNV
IIMGAEPDLFETNSSNISLRNNYMPSNHGFGSIAIVTFSPEYSFRFNDNS
MNEFIQDPALTLMHELIHSLHGLYGAKGITTKYTITQKQNPLITNIRGTN
IEEFLTFGGTDLNIITSAQSNDIYTNLLADYKKIASKLSKVQVSNPLLNP
YKDVFEAKYGLDKDASGIYSVNINKFNDIFKKLYSFTEFDLATKFQVKCR
QTYIGQYKYFKLSNLLNDSIYNISEGYNINNLKVNFRGQNANLNPRIITP
ITGRGLVKKIIRFCKNIVSVKGIRKSICIEINNGELFFVASENSYNDDNI
NTPKEIDDTVTSNNNYENDLDQVILNFNSESAPGLSDEKLNLTIQN
DAYIPKYDSNGTSDIEQHDVNELNVFFYLDAQKVPEGENNVNLTS
SIDTALLEQPKIYTFFSSEFINNVNKPVQAALFVSWIQQVLVDFTTE
ANQKSTVDKIADISIVVPYIGLALNIGNEAQKGNFKDALELLGAGILL
EFEPELLIPTILVFTIKSFLGSSDNKNKVIKAINNALKERDEKWKEV
YSFIVSNWMTKINTQFNKRKEQMYQALQNQVNAIKTIIESKYNSYTL
EEKNELTNKYDIKQIENELNQKVSIAMNNIDRFLTESSISYLMKLINE
VKINKLREYDENVKTYLLNYIIQHGSILGESQQELNSMVTDTLNNSIP
FKLSSYTDDKILISYFNKFFK

SEQ ID NO: 7 BoNT Type E H_{C} Fragment from Clostridium botulinum Strain Alaska E43, which is a Bont/E Subtype E₃ Neurotoxin)

RIKSSSVLNMRYKNDKYVDTSGYDSNININGDVYKYPTNKNQFGIYND
KLSEVNISQNDYIIYDNKYKNFSISFWVRIPNYDNKIVNVNNEYTIINCM
RDNNSGWKVSLNHNEIIWTLQDNAGINQKLAFNYGNANGISDYINKWIF
VTITNDRLGDSKLYINGNLIDQKSILNLGNIHVSDNILFKIVNCSYTRYI
GIRYFNIFDKELDETEIQTLYSNEPNTNILKDFWGNYLLYDKEYYLLNV
LKPNNFIDRRKDSTLSINNIRSTILLANRLYSGIKVKIQRVNNSSTNDNL
VRKNDQVYINFVASKTHLFPLYADTATTNKEKTIKISSSGNRFNQVVV
MNSVGNNCTMNFKNNNGNNIGLLGFKADTVVASTWYYTHMRDHTNSNG
CFWNFISEEHGWQEK

SEQ ID NO: 8 BoNT Type E Vaccine (Clostridium botulinum Strain Alaska E43 Containing Glu 213 to Gln, and His 216 to Tyr Mutations in the Light Chain to Negate Endopeptidase Activity and Trp 1224 to Leu and Tyr 1225 to Phe in the H_{C} Domain to Negate Receptor Binding Activity

MPKINSFNYNDPVNDRTILYIKPGGCQEFYKSFNIMKNIWIIPE
RNVIGTTPQDFHPPTSLKNGDSSYYDPNYLQSDEEKDRFLKIVTKIFNRINNNLSGGILL
EELSKANPYLGNDNTPDNQFHIGDASAVEIKFSNGSQHILLPNVIIMGAEPDLFET
NSSNISLRNNYMPSNHGFGSIAIVTFSPEYSFRFNDNSINEFIQDPALTLMHQLIYSL
HGLYGAKGITTTCIITQQQNPLITNRKGINIEEFLTFGGNDLNIITVAQYNDIYTNLL
NDYRKIASKLSKVQVSNPQLNPYKDIFQEKYGLDKDASGIYSVNINKFDDILKKLYSF
TEFDLATKFQVKCRETYIGQYKYFKLSNLLNDSIYNISEGYNINNLKVNFRGQNANLN
PRIIKPITGRGLVKKIIRFCKNIVSVKGIRKSICIEINNGELFFVASENSYNDDNINT
PKEIDDTVTSNNNYENDLDQVILNFNSESAPGLSDEKLNLTIQNDAYIPKYDSNGTSD
IEQHDVNELNVFFYLDAQKVPEGENNVNLTSSIDTALLEQPKIYTFFSSEFINNVNKP
VQAALFVSWIQQVLVDFTTEANQKSTVDKIADISIVVPYIGLALNIGNEAQKGNFKDA
LELLGAGILLEFEPELLIPTILVFTIKSFLGSSDNKNKVIKAINNALKERDEKWKEVY
SFIVSNWMTKINTQFNKRKEQMYQALQNQVNAIKTIIESKYNSYTLEEKNELTNKYDI
KQIENELNQKVSIAMNNIDRFLTESSISYLMKLINEVKINKLREYDENVKTYLLNYII
QHGSILGESQQELNSMVTDTLNNSIPFKLSSYTDDKILISYFNKFFK RIKSSSVLNMR
YKNDKYVDTSGYDSNININGDVYKYPTNKNQFGIYNDKLSEVNISQNDYIIYDNKYKN
FSISFWVRIPNYDNKIVNVNNEYTIINCMRDNNSGWKVSLNHNEIIWTLQDNAGINQK
LAFNYGNANGISDYINKWIFVTITNDRLGDSKLYINGNLIDQKSILNLGNIHVSDNIL
FKIVNCSYTRYIGIRYFNIFDKELDETEIQTLYSNEPNTNILKDFWGNYLLYDKEYYL
LNVLKPNNFIDRRKDSTLSINNIRSTILLANRLYSGIKVKIQRVNNSSTNDNLVRKND
QVYINFVASKTHLFPLYADTATTNKEKTIKISSSGNRFNQVVVMNSVGNNCTMNFKNN
NGNNIGLLGFKADTVVASTLFYTHMRDHTNSNGCFWNFISEEHGWQEK

### SEQ ID NO: 9 BoNT Type E Vaccine (Extended LH_{N} Fragment (2-1067) (Subtype E3))

MPKINSFNYNDPVNDRTILYIKPGGCQEFYKSFNIMKNIWIIPE
RNVIGTTPQDFHPPTSLKNGDSSYYDPNYLQSDEEKDRFLKIVTKIFNRINNNLSGGILL
EELSKANPYLGNDNTPDNQFHIGDASAVEIKFSNGSQHILLPNVIIMGAEPDLFET
NSSNISLRNNYMPSNHGFGSIAIVTFSPEYSFRFNDNSINEFIQDPALTLMHELIHSL
HGLYGAKGITTTCIITQQQNPLITNRKGINIEEFLTFGGNDLNIITVAQYNDIYTNLL
NDYRKIASKLSKVQVSNPQLNPYKDIFQEKYGLDKDASGIYSVNINKFDDILKKLYSF
TEFDLATKFQVKCRETYIGQYKYFKLSNLLNDSIYNISEGYNINNLKVNFRGQNANLN
PRIIKPITGRGLVKKIIRFCKNIVSVKGIRKSICIEINNGELFFVASENSYNDDNINT
PKEIDDTVTSNNNYENDLDQVILNFNSESAPGLSDEKLNLTIQNDAYIPKYDSNGTSD
IEQHDVNELNVFFYLDAQKVPEGENNVNLTSSIDTALLEQPKIYTFFSSEFINNVNKP
VQAALFVSWIQQVLVDFTTEANQKSTVDKIADISIVVPYIGLALNIGNEAQKGNFKDA
LELLGAGILLEFEPELLIPTILVFTIKSFLGSSDNKNKVIKAINNALKERDEKWKEVY
SFIVSNWMTKINTQFNKRKEQMYQALQNQVNAIKTIIESKYNSYTLEEKNELTNKYDI
KQIENELNQKVSIAMNNIDRFLTESSISYLMKLINEVKINKLREYDENVKTYLLNYII
QHGSILGESQQELNSMVTDTLNNSIPFKLSSYTDDKILISYFNKFFKRIKSSSVLNMR
YKNDKYVDTSGYDSNININGDVYKYPTNKNQFGIYNDKLSEVNISQNDYIIYDNKYKN
FSISFWVRIPNYDNKIVNVNNEYTIINCMRDNNSGWKVSLNHNEIIWTLQDNAGINQK
LAFNYGNANGISDYINKWIFVTITNDRLGDSKLYINGNLIDQKSILNLGNIHVSDNIL
FKIVNCSYTRYIGIRYFNIFDKELDETEIQTLYSNEP

### SEQ ID NO: 10 BoNT/A1 LH_{N} Sequence

MPFVNKQFNYKDPVNGVDIAYIKIPNVGQMQPVKAFKIHNKIWVIPERDTFTNPEEGDLNP
PPEAKQVPVSYYDSTYLSTDNEKDNYLKGVTKLFERIYSTDLGRMLLTSIVRGIPFWGGS
TIDTELKVIDTNCINVIQPDGSYRSEELNLVIIGPSADIIQFECKSFGHEVLNLTRNGYG
STQYIRFSPDFTFGFEESLEVDTNPLLGAGKFATDPAVTLAHQLIYAGHRLYGIAINPNR
VFKVNTNAYYEMSGLEVSFEELRTFGGHDAKFIDSLQENEFRLYYYNKFKDIASTLNKAK
SIVGTTASLQYMKNVFKEKYLLSEDTSGKFSVDKLKFDKLYKMLTEIYTEDNFVKFFKVL
NRKTYLNFDKAVFKINIVPKVNYTIYDGFNLRNTNLAANFNGQNTEINNMNFTKLKNFTG
LFEFYKLLCVRGIITSKTKSLDKGYNKALNDLCIKVNNWDLFFSPSEDNFTNDLNKGEEI
TSDTNIEAAEENISLDLIQQYYLTFNFDNEPENISIENLSSDIIGQLELMPNIERFPNGK
KYELDKYTMFHYLRAQEFEHGKSRIALTNSVNEALLNPSRVYTFFSSDYVKKVNKATEAA
MFLGWVEQLVYDFTDETSEVSTTDKIADITIIIPYIGPALNIGNMLYKDDFVGALIFSGA
VILLEFIPEIAIPVLGTFALVSYIANKVLTVQTIDNALSKRNEKWDEVYKYIVTNWLAKV
NTQIDLIRKKMKEALENQAEATKAIINYQYNQYTEEEKNNINFNIDDLSSKLNESINKAM
ININKFLNQCSVSYLMNSMIPYGVKRLEDFDASLKDALLKYIYDNRGTLIGQVDRLKDKV
NNTLSTDIPFQLSKYVDNQRLLSTFTEYIK

### SEQ ID NO: 11 BoNT/A2 LH_{N} Sequence

MPFVNKQFNYKDPVNGVDIAYIKIPNAGQMQPVKAFKIHNKIWVIPERDTFTNPEEGDLNP
PPEAKQVPVSYYDSTYLSTDNEKDNYLKGVTKLFERIYSTDLGRMLLTSIVRGIPFWGGS
TIDTELKVIDTNCINVIQPDGSYRSEELNLVIIGPSADIIQFECKSFGHDVLNLTRNGYG
STQYIRFSPDFTFGFEESLEVDTNPLLGAGKFATDPAVTLA HQLIYAEHRLYGIAINPNR
VFKVNTNAYYEMSGLEVSFEELRTFGGHDAKFIDSLQENEFRLYYYNKFKDVASTLNKAK
SIIGTTASLQYMKNVFKEKYLLSEDTSGKFSVDKLKFDKLYKMLTEIYTEDNFVNFFKVI
NRKTYLNFDKAVFRINIVPDENYTIKDGFNLKGANLSTNFNGQNTEINSRNFTRLKNFTG
LFEFYKLLCVRGIIPFKTKSLDEGYNKALNDLCIKVNNWDLFFSPSEDNFTNDLDKVEEI
TADTNIEAAEENISLDLIQQYYLTFDFDNEPENISIENLSSDIIGQLEPMPNIERFPNGK
KYELDKYTMFHYLRAQEFEHGDSRIILTNSAEEALLKPNVAYTFFSSKYVKKINKAVEAF
MFLNWAEELVYDFTDETNEVTTMDKIADITIIVPYIGPALNIGNMLSKGEFVEAIIFTGV
VAMLEFIPEYALPVFGTFAIVSYIANKVLTVQTINNALSKRNEKWDEVYKYTVTNWLAKV
NTQIDLIREKMKKALENQAEATKAIINYQYNQYTEEEKNNINFNIDDLSSKLNESINSAM
ININKFLDQCSVSYLMNSMIPYAVKRLKDFDASVRDVLLKYIYDNRGTLVLQVDRLKDEV
NNTLSADIPFQLSKYVDNKKLLSTFTEYIK

### SEQ ID NO: 12 BoNT/A3 LH_{N} Sequence

MPFVNKPFNYRDPGNGVDIAYIKIPNAGQMQPVKAFKIHEGVWVIPERDTFTNPEEGDLNPPPEA
KQVPVSYYDSTYLSTDNEKDNYLKGVIKLFDRIYSTGLGRMLLSFIVKGIPFWGGSTIDTELKVIDT
NCINVIEPGGSYRSEELNLVITGPSADIIQFECKSFGHDVFNLTRNGYGSTQYIRFSPDFTFGFEES
LEVDTNPLLGAGTFATDPAVTLAHQLIYAAHRLYGIAINPNRVLKVKTNAYYEMSGLEVSFEELRT
FGGNDTNFIDSLWQKKFSRDAYDNLQNIARILNEAKTIVGTTTPLQYMKNIFIRKYFLSEDASGKIS
VNKAAFKEFYRVLTRGFTELEFVNPFKVINRKTYLNFDKAVFRINIVPDENYTINEGFNLEGANSN
GQNTEINSRNFTRLKNFTGLFEFYKLLCVRGIIPFKTKSLDEGYNKALNYLCIKVNNWDLFFSPSE
DNFTNDLDKVEEITADTNIEAAEENISSDLIQQYYLTFDFDNEPENISIENLSSDIIGQLEPMPNIERF
PNGKKYELDKYTMFHYLRAQEFEHGDSRIILTNSAEEALLKPNVAYTFFSSKYVKKINKAVEAVIFL
SWAEELVYDFTDETNEVTTMDKIADITIIVPYIGPALNIGNMVSKGEFVEAILFTGVVALLEFIPEYSL
PVFGTFAIVSYIANKVLTVQTINNALSKRNEKWDEVYKYTVTNWLAKVNTQIDLIREKMKKALENQ
AEATRAIINYQYNQYTEEEKNNINFNIDDLSSKLNRSINRAMININKFLDQCSVSYLMNSMIPYAVK
RLKDFDASVRDVLLKYIYDNRGTLILQVDRLKDEVNNTLSADIPFQLSKYVNDKKLLSTFTEYIK

### SEQ ID NO: 13 BoNT/A4 LH_{N} Sequence

MPLVNQQINYYDPVNGVDIAYIKIPNAGKMQPVKAFKIHNKVWVIPERDIFTNPEEVDLNPPPEAK
QVPISYYDSAYLSTDNEKDNYLKGVIKLFERIYSTDLGRMLLISIVRGIPFWGGGKIDTELKVIDTNC
INIIQLDDSYRSEELNLAIIGPSANIIESQCSSFRDDVLNLTRNGYGSTQYIRFSPDFTNGFEESLEV
DTNPLLGAGKFAQDPAVALAHQLIYAEHRLYGIAINTNRVFKVNTNAYYEMAGLEVSLEELITFGG
NDAKFIDSLQKKEFSLYYYNKFKDIASTLNKAKSIVGTTASLQYMKNVFKEKYLLSEDATGKFLVD
RLKFDELYKLLTEIYTEDNFVKFFKVLNRKTYLNFDKAVFKINIVPDVNYTIHDGFNLRNTNLAANF
NGQNIEINNKNFDKLKNFTGLFEFYKLLCVRGIITSKTKSLDEGYNKALNELCIKVNNWDLFFSPSE
DNFTNDLDKVEEITSDTNIEAAEENISLDLIQQYYLNFNFDNEPENTSIENLSSDIIGQLEPMPNIER
FPNGKKYELNKYTMFHYLRAQEFKHSNSRIILTNSAKEALLKPNIVYTFFSSKYIKAINKAVEAVTF
VNWIENLVYDFTDETNEVSTMDKIADITIVIPYIGPALNIGNMIYKGEFVEAIIFSGAVILLEIVPEIALP
VLGTFALVSYVSNKVLTVQTIDNALSKRNEKWDEVYKYIVTNWLAIVNTQINLIREKMKKALENQA
EATKAIINYQYNQYTEEEKNNINFNIDDLSSKLNESINSAMININKFLDQCSVSYLMNSMIPYAVKRL
KDFDASVRDVLLKYIYDNRGTLIGQVNRLKDKVNNTLSADIPFQLSKYVDNKKLLSTFTEYIK

### SEQ ID NO: 14 BoNT/B1 LH_{N} Sequence

MSVTINNFNYNDPIDNDNIIMMEPPFARGTGRYYKAFKITDRIWIIPERYTFGYKPEDFNKSSGIFN
RDVCEYYDPDYLNTNDKKNIFLQTMIKLFNRIKSKPLGEKLLEMIINGIPYLGDRRVPLEEFNTNIAS
VTVNKLISNPGEVERKKGIFANLIIFGPGPVLNENETIDIGIQNHFASREGFGGIMQMKFCPEYVSV
FNNVQENKGASIFNRRGYFSDPALILMHQLIYVLHGLYGIKVDDLPIVPNEKKFFMQSTDAIQAEEL
YTFGGQDPSIITPSTDKSIYDKVLQNFRGIVDRLNKVLVCISDPNININIYKNKFKDKYKFVEDSEGK
YSIDVESFDKLYKSLMFGFTETNIAENYKIKTRASYFSDSLPPVKIKNLLDNEIYTIEEGFNISDKDM
EKEYRGQNKAINKQAYEEISKEHLAVYKIQMCKSVKAPGICIDVDNEDLFFIADKNSFSDDLSKNE
RIEYNTQSNYIENDFPINELILDTDLISKIELPSENTESLTDFNVDVPAYEKQPAIKKIFTDENTIFQYL
YSQTFPLDIRDISLTSSFDDALLFSNKVYSFFSMDYIKTANKVVEAGLFAGWVKQIVNDFVIEANKS
NTMDKIADISLIVPYIGLALNVGNETAKGNFENAFEIAGASILLEFIPELLIPVVGAFLLESYIDNKNKII
KTIDNALTKRNEKWSDMYGLIVAQWLSTVNTQFYTIKEGMYKALNYQAQALEEIIKYRYNIYSEKE
KSNINIDFNDINSKLNEGINQAIDNINNFINGCSVSYLMKKMIPLAVEKLLDFDNTLKKNLLNYIDEN
KLYLIGSAEYEKSKVNKYLKTIMPFDLSIYTNDTILIEMFNKYNS

### SEQ ID NO: 15 BoNT/B2 LH_{N} Sequence

MPVTINNFNYNDPIDNNNIIMMEPPFARGTGRYYKAFKITDRIWIIPERYTFGYKPEDFNKSSGIFN
RDVCEYYDPDYLNTNDKKNIFLQTMIKLFNRIKSKPLGEKLLEMIINGIPYLGDRRVPLEEFNTNIAS
VTVNKLISNPGEVERKKGIFANLIIFGPGPVLNENETIDIGIQNHFASREGFGGIMQMKFCPEYVSV
FNNVQENKGASIFNRRGYFSDPALILMHQLIYVLHGLYGIKVDDLPIVPNEKKFFMQSTDAIQAEEL
YTFGGQDPSIITPSTDKSIYDKVLQNFRGIVDRLNKVLVCISDPNININIYKNKFKDKYKFVEDSEGK
YSIDVESFDKLYKSLMFGFTETNIAENYKIKTRASYFSDSLPPVKIKNLLDNEIYTIEEGFNISDKNM
EKEYRGQNKAINKQAYEEISKEHLAVYKIQMCKSVRAPGICIDVDNEDLFFIADKNSFSDDLSKNE
RIEYDTQSNYIENRSSIDELILDTNLISKIELPSENTESLTDFNVDVPVYEKQPAIKKIFTDENTIFQYL
YSQTFPLDIRDISLTSSFDDALLFSKKVYSFFSMDYIKTANKVVEAGLFAGWVKQIVDDFVIEANKS
STMDKIADISLIVPYIGLALNVGNETAKGNFENAFEIAGASILLEFIPELLIPVVGAFLLESYIDNKNKII
KTIDNALTKRDEKWIDMYGLIVAQWLSTVNTQFYTIKEGMYKALNYQAQALEEIIKYKYNIYSEKEK
SNINIDFNDINSKLNEGINQAIDNINNFINECSVSYLMKKMIPLAVEKLLDFDNTLKKNLLNYIDENKL
YLIGSAEYEKSKVDKHLKTIIPFDLSKYTNNTILIEIFNKYNS

### SEQ ID NO: 16 BoNT/B3 LH_{N} Sequence

MPVTINNFNYNDPIDNDNIIMMEPPFARGTGRYYKAFKITDRIWIIPERYTFGYKPEDFNKSSGIFN
RDVCEYYDPDYLNTNDKKNIFLQTMIKLFNRIKSKPLGEKLLEMIINGIPYLGDRRVPLEEFNTNIAS
VTVNKLISNPGEVERKKGIFANLIIFGPGPVLNENETIDIGIQNHFASREGFGGIMQMKFCPEYVSV
FNNVQENKGASIFNRRGYFSDPALILMHQLIYVLHGLYGIKVDDLPIVPNEKKFFMQSTDAIQAEEL
YTFGGQDPRIITPSTDKSIYDKVLQNFRGIVDRLNKVLVCISDPNININIYKNKFKDKYKFVEDSEGK
YSIDVESFDKLYKSLMFGFTETNIAENYKIKTRASYFSDSLPPVKIKNLLDNEIYTIEEGFNISDKNM
EKEYRGQNKAINKQAYEEISKEHLAVYKIQMCKSVRAPGICIDVDNEDLFFIADKNSFSDDLSKNE
RIEYDTQSNYIENRSSIDELILDTNLISKIELPSENTESLTDFNVDVPVYEKQPAIKKIFTDENTIFQYL
YSQTFPLDIRDISLTSSFDDALLFSNKVYSFFSMDYIKTANKVVEAGLFAGWVKQIVDDFVIEANKS
STMDKIADISLIVPYIGLALNVGNETAKGNFENAFEIAGASILLEFIPELLIPVVGAFLLESYIDNKNKII
KTIDNALTKRDEKWIDMYGLIVAQWLSTVNTQFYTIKEGMYKALNYQAQALEEIIKYKYNIYSEKEK
SNINIDFNDINSKLNEGINQAIDNINNFINECSVSYLMKKMIPLAVEKLLDFDNTLKKNLLNYIDENKL
YLIGSAEYEKSKVDKHLKTIIPFDLSMYTNNTILIEIFNKYNS

### SEQ ID NO: 17 BoNT/B4 LH_{N} Sequence

MPVTINNFNYNDPIDNDNIIMMEPPFARGTGRYYKAFKITDRIWIIPERYTFGYKPEDFNKSSGIFN
RDVCEYYDPDYLNTNDKKNIFLQTMIKLFNRIKSKPLGEKLLEMIINGIPYLGDRRVPLEEFNTNIAS
VTVNKLISNPGEVEQKKGIFANLIIFGPGPVLNENETIDIGIQNHFASREGFGGIMQMKFCPEYVSV
FNNVQENKGASIFNRRGYFSDPALILMHQLIYVLHGLYGIKVDDLPIVPNEKKFFMQSTDTIQAEEL
YTFGGQDPSIISPSTDKSIYDKVLQNFRGIVDRLNKVLVCISDPNININIYKNKFKDKYKFVEDSEGK
YSIDVESFNKLYKSLMFGFTEINIAENYKIKTRASYFSDSLPPVKIKNLLDNEIYTIEEGFNISDKNM
GKEYRGQNKAINKQAYEEISKEHLAVYKIQMCKSVKVPGICIDVDNENLFFIADKNSFSDDLSKNE
RVEYNTQNNYIGNDFPINELILDTDLISKIELPSENTESLTDFNVDVPVYEKQPAIKKVFTDENTIFQ
YLYSQTFPLNIRDISLTSSFDDALLVSSKVYSFFSMDYIKTANKVVEAGLFAGWVKQIVDDFVIEAN
KSSTMDKIADISLIVPYIGLALNVGNETAKGNFESAFEIAGSSILLEFIPELLIPVVGVFLLESYIDNKN
KIIKTIDNALTKRVEKWIDMYGLIVAQWLSTVNTQFYTIKEGMYKALNYQAQALEEIIKYKYNIYSEE
EKSNININFNDINSKLNDGINQAMDNINDFINECSISYLMKKMIPLAVKKLLDFDNTLKKNLLNYIDE
NKLYLIGSVEDEKSKVDKYLKTIIPFDLSTYTNNEILIKIFNKYNS

### SEQ ID NO: 18 BoNT/F1 LH_{N} Sequence

MPVVINSFNYNDPVNDDTILYMQIPYEEKSKKYYKAFEIMRNVWIIPERNTIGTDPSDFDPPASLE
NGSSAYYDPNYLTTDAEKDRYLKTTIKLFKRINSNPAGEVLLQEISYAKPYLGNEHTPINEFHPVT
RTTSVNIKSSTNVKSSIILNLLVLGAGPDIFENSSYPVRKLMDSGGVYDPSNDGFGSINIVTFSPEY
EYTFNDISGGYNSSTESFIADPAISLAHQLIYALHGLYGARGVTYKETIKVKQAPLMIAEKPIRLEEF
LTFGGQDLNIITSAMKEKIYNNLLANYEKIATRLSRVNSAPPEYDINEYKDYFQWKYGLDKNADGS
YTVNENKFNEIYKKLYSFTEIDLANKFKVKCRNTYFIKYGFLKVPNLLDDDIYTVSEGFNIGNLAVN
NRGQNIKLNPKIIDSIPDKGLVEKIVKFCKSVIPRKGTKAPPRLCIRVNNRELFFVASESSYNENDIN
TPKEIDDTTNLNNNYRNNLDEVILDYNSETIPQISNQTLNTLVQDDSYVPRYDSNGTSEIEEHNVV
DLNVFFYLHAQKVPEGETNISLTSSIDTALSEESQVYTFFSSEFINTINKPVHAALFISWINQVIRDF
TTEATQKSTFDKIADISLVVPYVGLALNIGNEVQKENFKEAFELLGAGILLEFVPELLIPTILVFTIKSF
IGSSENKNKIIKAINNSLMERETKWKEIYSWIVSNWLTRINTQFNKRKEQMYQALQNQVDAIKTVIE
YKYNNYTSDERNRLESEYNINNIREELNKKVSLAMENIERFITESSIFYLMKLINEAKVSKLREYDE
GVKEYLLDYISEHRSILGNSVQELNDLVTSTLNNSIPFELSSYTNDKILILYFNKLYK

### SEQ ID NO: 19 BoNT/F2 LH_{N} Sequence

MPVAINSFNYNDPVNDDTILYMQIPYEEKSKKYYKAFEIMRNVWIIPERNTIGTNPSDFDPPASLK
NGSSAYYDPNYLTTDAEKDRYLKTTIKLFKRINSNPAGKVLLQEISYAKPYLGNDHTPIDEFSPVT
RTTSVNIKLSTNVESSMLLNLLVLGAGPDIFESCCYPVRKLIDPDVVYDPSNYGFGSINIVTFSPEY
EYTFNDISGGHNSSTESFIADPAISLAHQLIYALHGLYGARGVTYEETIEVKQAPLMIAEKPIRLEEF
LTFGGQDLNIITSAMKEKIYNNLLANYEKIATRLSEVNSAPPEYDINEYKDYFQWKYGLDKNADGS
YTVNENKFNEIYKKLYSFTESDLANKFKVKCRNTYFIKYEFLKVPNLLDDDIYTVSEGFNIGNLAVN
NRGQSIKLNPKIIDSIPDKGLVEKIVKFCKSVIPRKGTKAPPRLCIRVNNSELFFVASESSYNENDIN
TPKEIDDTTNLNNNYRNNLDEVILDYNSQTIPQISNRTLNTLVQDNSYVPRYDSNGTSEIEEYDVV
DFNVFFYLHAQKVPEGETNISLTSSIDTALLEESKDIFFSSEFIDTINKPVNAALFIDWISKVIRDFTT
EATQKSTVDKIADISLIVPYVGLALNIIIEAEKGNFEEAFELLGVGILLEFVPELTIPVILVFTIKSYIDSY
ENKNKAIKAINNSLIEREAKWKEIYSWIVSNWLTRINTQFNKRKEQMYQALQNQVDAIKTAIEYKY
NNYTSDEKNRLESEYNINNIEEELNKKVSLAMKNIERFMTESSISYLMKLINEAKVGKLKKYDNHV
KSDLLNYILDHRSILGEQTNELSDLVTSTLNSSIPFELSSYTNDKILIIYFNRLYK

SEQ ID NO: 20 BoNT/F3 (barati) LH_{N} Sequence

MPVNINNFNYNDPINNTTILYMKMPYYEDSNKYYKAFEIMDNVWIIPERNIIGKKPSDFYPPISLDS
GSSAYYDPNYLTTDAEKDRFLKTVIKLFNRINSNPAGQVLLEEIKNGKPYLGNDHTAVNEFCANN
RSTSVEIKESNGTTDSMLLNLVILGPGPNILECSTFPVRIFPNNIAYDPSEKGFGSIQLMSFSTEYE
YAFNDNTDLFIADPAISLAHQLIYVLHGLYGAKGVTNKKVIEVDQGALMAAEKDIKIEEFITFGGQD
LNIITNSTNQKIYVILLSNYTAIASRLSQVNANNSALNTTYYKNFFQWKYGLDQDSNGNYTVNISKF
NAIYKKLFSFTECDLAQKFQVKNRSNYLFHFKPFRLLDLLDDNIYSISEGFNIGSLRVNNNGQNINL
NSRIVGPIPDNGLVERFVGLCKSIVSKKGTKNSLCIKVNNRDLFFVASESSYNENGINSPKEIDDTT
ITNNNYKKNLDEVILDYNSDAIPNLSSRLLNTTAQNDSYVPKYDSNGTSEIKEYTVDKLNVFFYLYA
QKAPEGESAISLTSSVNTALLDASKVYTFFSSDFINTVNKPVQAALFISWIQQVINDFTTEATQKSTI
DKIADISLIVPYVGLALNIGNEVQKGNFKEAIELLGAGILLEFVPELLIPTILVFTIKSFINSDDSKNKIIK
AINNALRERELKWKEVYSWIVSNWLTRINTQFNKRKEQMYQALQNQVDGIKKIIEYKYNNYTLDE
KNRLRAEYNIYSIKEELNKKVSLAMQNIDRFLTESSISYLMKLINEAKINKLSEYDKRVNQYLLNYIL
ENSSTLGTSSVPELNNLVSNTLNNSIPFELSEYTNDKILIHILIRFYK

1. A vaccine composition, comprising a botulinum serotype E (BoNT/E) peptide, said peptide comprising an amino acid sequence having at least 90% sequence identity to amino acid residues 100-750 of SEQ ID NO: 1:
(i) wherein said BoNT/E peptide lacks a functional H_{C} of a clostridial neurotoxin heavy chain; and
(i) wherein the BoNT/E peptide contains one or more of H142, G176, S198, I199, T230, C231, I232, Q236, R244, K245, I247, N258, V265, Y268, N277, R280, Q295, I302, Q304, E305, D325, L328, A340, E349, K397, and L773.

2-3. (canceled)

4. The composition according to claim 1, wherein the peptide comprises at least 90% sequence identity to amino acid residues 50-800 of SEQ ID NO: 1.

5. The composition according to claim 1, wherein the peptide comprises any four amino acid residues selected from H142, G176, S198, I199, T230, C231, I232, Q236, R244, K245, I247, N258, V265, Y268, N277, R280, Q295, I302, Q304, E305, D325, L328, A340, E349, K397, and L773.

6. The composition according to claim 1, wherein the peptide comprises any eight amino acid residues selected from H142, G176, S198, I199, T230, C231, I232, Q236, R244, K245, I247, N258, V265, Y268, N277, R280, Q295, I302, Q304, E305, D325, L328, A340, E349, K397, and L773.

7-9. (canceled)

10. The composition according to claim 1, wherein the peptide comprises H142, G176, S198, I199, T230, C231, I232, Q236, R244, K245, I247, N258, V265, Y268, N277, R280, Q295, I302, Q304, E305, D325, L328, A340, E349, K397, and L773.

11-19. (canceled)

20. The composition according to claim 1, wherein the peptide further comprises a mutation which reduces zinc co-ordination at the active site.

21. The composition according to claim 1, wherein the peptide comprises amino acid residues 2-845 of SEQ ID NO: 1, 2 or 3.

22. (canceled)

23. The composition according to claim 1, wherein the peptide further comprises a protease cleavage site located at a position that corresponds to a position between residues 411 and 426 of SEQ ID NO: 1.

24. (canceled)

25. The composition according to claim 1, wherein the peptide comprises an amino acid sequence having at least 90% sequence identity to SEQ ID NO: 9.

26. (canceled)

27. The composition according to claim 1, said composition further comprising one or more different non-serotype E BoNT peptide(s) that lack a functional H_{C} of a clostridial neurotoxin heavy chain.

28-32. (canceled)

33. The composition according to claim 27, wherein the BoNT peptide(s) has been treated with a chemical modifying agent to improve the peptide(s) ability to stimulate an immune response.

34-35. (canceled)

36. The composition according to claim 33, wherein the chemical modifying agent is formaldehyde.

37. (canceled)

38. A method for preparing a composition according to claim 33, comprising contacting the BoNT peptide(s) with the chemical modifying agent for a period of less than three days.

39. The method according to claim 38, wherein treatment of the BoNT peptide(s) with the chemical modifying agent occurs at a molar ratio (chemical modifying agent to BoNT peptide(s)) of less than 50:1.

40. The method according to claim 39, wherein treatment of the BoNT peptide(s) with the chemical modifying agent occurs in a reaction mix at a ratio (chemical modifying agent to BoNT peptide(s)) of less than 2%.

41. The method according to claim 38, wherein the chemical modifying agent is formaldehyde.

42. A method of stimulating an immune response in a mammal, comprising administering to the mammal an effective amount of the composition according to claim 1.

43. A method of protecting against BoNT poisoning in a mammal, comprising administering to the mammal an effective amount of the composition according to claim 1.

44-47. (canceled)

48. A method of stimulating an immune response in a mammal, comprising administering to the mammal an effective amount of the composition according to claim 33.

49. A method of protecting against BoNT poisoning in a mammal, comprising administering to the mammal an effective amount of the composition according to claim 33.


SEQUENCE LISTING (46 sequences)

SEQ ID NO: 1 - protein, 845 aa, Artificial Sequence
  Description of Artificial Sequence Synthetic polypeptide
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHELIHSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFK

SEQ ID NO: 2 - protein, 845 aa, Artificial Sequence
  Description of Artificial Sequence Synthetic polypeptide
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHQLIHSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFK

SEQ ID NO: 3 - protein, 845 aa, Artificial Sequence
  Description of Artificial Sequence Synthetic polypeptide
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHQLIYSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFK

SEQ ID NO: 4 - protein, 825 aa, Artificial Sequence
  Description of Artificial Sequence Synthetic polypeptide
MPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHQLIYSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKI

SEQ ID NO: 5 - protein, 825 aa, Artificial Sequence; identical to SEQ ID NO: 4
  Description of Artificial Sequence Synthetic polypeptide

SEQ ID NO: 6 - protein, 845 aa, Artificial Sequence
  Description of Artificial Sequence Synthetic polypeptide
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGIQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFK

SEQ ID NO: 7 - protein, 407 aa, Artificial Sequence
  Description of Artificial Sequence Synthetic polypeptide
RIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 8 - protein, 1252 aa, Artificial Sequence
  Description of Artificial Sequence Synthetic polypeptide
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHQLIYSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTLFYTHMRDHTNSNGCFWNFISEEHGWQEK

SEQ ID NO: 9 - protein, 1067 aa, Artificial Sequence
  Description of Artificial Sequence Synthetic polypeptide
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHELIHSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEP

SEQ ID NO: 10 - protein, 871 aa, Artificial Sequence
  Description of Artificial Sequence Synthetic polypeptide
MPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIK

SEQ ID NO: 11 - protein, 871 aa, Artificial Sequence
  Description of Artificial Sequence Synthetic polypeptide
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHQLIYAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIK

SEQ ID NO: 12 - protein, 867 aa, Artificial Sequence
  Description of Artificial Sequence Synthetic polypeptide
MPFVNKPFNYRDPGNGVDIAYIKIPNAGQMQPVKAFKIHEGVWVIPERDTFTNPEEGDLNPPPEAKQVPVSYYDSTYLSTDNEKDNYLKGVIKLFDRIYSTGLGRMLLSFIVKGIPFWGGSTIDTELKVIDTNCINVIEPGGSYRSEELNLVITGPSADIIQFECKSFGHDVFNLTRNGYGSTQYIRFSPDFTFGFEESLEVDTNPLLGAGTFATDPAVTLAHQLIYAAHRLYGIAINPNRVLKVKTNAYYEMSGLEVSFEELRTFGGNDTNFIDSLWQKKFSRDAYDNLQNIARILNEAKTIVGTTTPLQYMKNIFIRKYFLSEDASGKISVNKAAFKEFYRVLTRGFTELEFVNPFKVINRKTYLNFDKAVFRINIVPDENYTINEGFNLEGANSNGQNTEINSRNFTRLKNFTGLFEFYKLLCVRGIIPFKTKSLDEGYNKALNYLCIKVNNWDLFFSPSEDNFTNDLDKVEEITADTNIEAAEENISSDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAVIFLSWAEELVYDFTDETNEVTTMDKIADITIIVPYIGPALNIGNMVSKGEFVEAILFTGVVALLEFIPEYSLPVFGTFAIVSYIANKVLTVQTINNALSKRNEKWDEVYKYTVTNWLAKVNTQIDLIREKMKKALENQAEATRAIINYQYNQYTEEEKNNINFNIDDLSSKLNRSINRAMININKFLDQCSVSYLMNSMIPYAVKRLKDFDASVRDVLLKYIYDNRGTLILQVDRLKDEVNNTLSADIPFQLSKYVNDKKLLSTFTEYIK

SEQ ID NO: 13 - protein, 871 aa, Artificial Sequence
  Description of Artificial Sequence Synthetic polypeptide
MPLVNQQINYYDPVNGVDIAYIKIPNAGKMQPVKAFKIHNKVWVIPERDIFTNPEEVDLNPPPEAKQVPISYYDSAYLSTDNEKDNYLKGVIKLFERIYSTDLGRMLLISIVRGIPFWGGGKIDTELKVIDTNCINIIQLDDSYRSEELNLAIIGPSANIIESQCSSFRDDVLNLTRNGYGSTQYIRFSPDFTVGFEESLEVDTNPLLGAGKFAQDPAVALAHQLIYAEHRLYGIAINTNRVFKVNTNAYYEMAGLEVSLEELITFGGNDAKFIDSLQKKEFSLYYYNKFKDIASTLNKAKSIVGTTASLQYMKNVFKEKYLLSEDATGKFLVDRLKFDELYKLLTEIYTEDNFVKFFKVLNRKTYLNFDKAVFKINIVPDVNYTIHDGFNLRNTNLAANFNGQNIEINNKNFDKLKNFTGLFEFYKLLCVRGIITSKTKSLDEGYNKALNELCIKVNNWDLFFSPSEDNFTNDLDKVEEITSDTNIEAAEENISLDLIQQYYLNFNFDNEPENTSIENLSSDIIGQLEPMPNIERFPNGKKYELNKYTMFHYLRAQEFKHSNSRIILTNSAKEALLKPNIVYTFFSSKYIKAINKAVEAVTFVNWIENLVYDFTDETNEVSTMDKIADITIVIPYIGPALNIGNMIYKGEFVEAIIFSGAVILLEIVPEIALPVLGTFALVSYVSNKVLTVQTIDNALSKRNEKWDEVYKYIVTNWLAIVNTQINLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLIGQVNRLKDKVNNTLSADIPFQLSKYVDNKKLLSTFTEYIK

SEQ ID NO: 14 - protein, 858 aa, Artificial Sequence
  Description of Artificial Sequence Synthetic polypeptide
MSVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHQLIYVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPAYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNS

SEQ ID NO: 15 - protein, 858 aa, Artificial Sequence
  Description of Artificial Sequence Synthetic polypeptide
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHQLIYVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKIFTDENTIFQYLYSQTFPLDIRDISLTSSFDDALLFSKKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNINIDFNDINSKLNEGINQAIDNINNFINECSVSYLMKKMIPLAVEKLLDFDNTLKKNLLNYIDENKLYLIGSAEYEKSKVDKHLKTIIPFDLSKYTNNTILIEIFNKYNS

SEQ ID NO: 16 - protein, 858 aa, Artificial Sequence
  Description of Artificial Sequence Synthetic polypeptide
MPVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHQLIYVLHGLYGIKVDDLPIVPNEKKFFMQSTDAIQAEELYTFGGQDPRIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNINIDFNDINSKLNEGINQAIDNINNFINECSVSYLMKKMIPLAVEKLLDFDNTLKKNLLNYIDENKLYLIGSAEYEKSKVDKHLKTIIPFDLSMYTNNTILIEIFNKYNS

SEQ ID NO: 17 - protein, 858 aa, Artificial Sequence
  Description of Artificial Sequence Synthetic polypeptide
MPVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVEQKKGIFANLIIFGPGPVLNENETIDIGIQNHFASREGFGGIMQMKFCPEYVSVFNNVQENKGASIFNRRGYFSDPALILMHQLIYVLHGLYGIKVDDLPIVPNEKKFFMQSTDTIQAEELYTFGGQDPSIISPSTDKSIYDKVLQNFRGIVDRLNKVLVCISDPNININIYKNKFKDKYKFVEDSEGKYSIDVESFNKLYKSLMFGFTEINIAENYKIKTRASYFSDSLPPVKIKNLLDNEIYTIEEGFNISDKNMGKEYRGQNKAINKQAYEEISKEHLAVYKIQMCKSVKVPGICIDVDNENLFFIADKNSFSDDLSKNERVEYNTQNNYIGNDFPINELILDTDLISKIELPSENTESLTDFNVDVPVYEKQPAIKKVFTDENTIFQYLYSQTFPLNIRDISLTSSFDDALLVSSKVYSFFSMDYIKTANKVVEAGLFAGWVKQIVDDFVIEANKSSTMDKIADISLIVPYIGLALNVGNETAKGNFESAFEIAGSSILLEFIPELLIPVVGVFLLESYIDNKNKIIKTIDNALTKRVEKWIDMYGLIVAQWLSTVNTQFYTIKEGMYKALNYQAQALEEIIKYKYNIYSEEEKSNININFNDINSKLNDGINQAMDNINDFINECSISYLMKKMIPLAVKKLLDFDNTLKKNLLNYIDENKLYLIGSVEDEKSKVDKYLKTIIPFDLSTYTNNEILIKIFNKYNS

SEQ ID NO: 18 - protein, 864 aa, Artificial Sequence
  Description of Artificial Sequence Synthetic polypeptide
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHQLIYALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYK

SEQ ID NO: 19 - protein, 863 aa, Artificial Sequence
  Description of Artificial Sequence Synthetic polypeptide
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHQLIYALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKGTKAPPRLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELSSYTNDKILIIYFNRLYK

SEQ ID NO: 20 - protein, 855 aa, Artificial Sequence
  Description of Artificial Sequence Synthetic polypeptide
MPVNINNFNYNDPINNTTILYMKMPYYEDSNKYYKAFEIMDNVWIIPERNIIGKKPSDFYPPISLDSGSSAYYDPNYLTTDAEKDRFLKTVIKLFNRINSNPAGQVLLEEIKNGKPYLGNDHTAVNEFCANNRSTSVEIKESNGTTDSMLLNLVILGPGPNILECSTFPVRIFPNNIAYDPSEKGFGSIQLMSFSTEYEYAFNDNTDLFIADPAISLAHQLIYVLHGLYGAKGVTNKKVIEVDQGALMAAEKDIKIEEFITFGGQDLNIITNSTNQKIYVILLSNYTAIASRLSQVNRNNSALNTTYYKNFFQWKYGLDQDSNGNYTVNISKFNAIYKKLFSFTECDLAQKFQVKNRSNYLFHFKPFRLLDLLDDNIYSISEGFNIGSLRVNNNGQNINLNSRIVGPIPDNGLVERFVGLCKSIVSKKGTKNSLCIKVNNRDLFFVASESSYNENGINSPKEIDDTTITNNNYKKNLDEVILDYNSDAIPNLSSRLLNTTAQNDSYVPKYDSNGTSEIKEYTVDKLNVFFYLYAQKAPEGESAISLTSSVNTALLDASKVYTFFSSDFINTVNKPVQAALFISWIQQVINDFTTEATQKSTIDKIADISLIVPYVGLALNIGNEVQKGNFKEAIELLGAGILLEFVPELLIPTILVFTIKSFINSDDSKNKIIKAINNALRERELKWKEVYSWIVSNWLTRINTQFNKRKEQMYQALQNQVDGIKKIIEYKYNNYTLDEKNRLRAEYNIYSIKEELNKKVSLAMQNIDRFLTESSISYLMKLINEAKINKLSEYDKRVNQYLLNYILENSSTLGTSSVPELNNLVSNTLNNSIPFELSEYTNDKILIHILIRFYK

SEQ ID NO: 21 - protein, 6 aa, Artificial Sequence
  Description of Artificial Sequence Synthetic peptide
ASTWYY

SEQ ID NO: 22 - protein, 6 aa, Artificial Sequence
  Description of Artificial Sequence Synthetic peptide
ASTLFY

SEQ ID NO: 23 - protein, 6 aa, Artificial Sequence
  Description of Artificial Sequence Synthetic peptide
ASTLYY

SEQ ID NO: 24 - protein, 6 aa, Artificial Sequence
  Description of Artificial Sequence Synthetic peptide
ASTWFY

SEQ ID NO: 25 - protein, 5 aa, Artificial Sequence
  Description of Artificial Sequence Synthetic peptide
HELIH

SEQ ID NO: 26 - protein, 5 aa, Artificial Sequence
  Description of Artificial Sequence Synthetic peptide
HQLIY

SEQ ID NO: 27 - protein, 5 aa, Artificial Sequence
  Description of Artificial Sequence Synthetic peptide
HALIY

SEQ ID NO: 28 - protein, 5 aa, Artificial Sequence
  Description of Artificial Sequence Synthetic peptide
HQLIH

SEQ ID NO: 29 - protein, 6 aa, Artificial Sequence
  Description of Artificial Sequence Synthetic peptide
SNWYNR

SEQ ID NO: 30 - protein, 6 aa, Artificial Sequence
  Description of Artificial Sequence Synthetic peptide
SNLFNR

SEQ ID NO: 31 - protein, 6 aa, Artificial Sequence
  Description of Artificial Sequence Synthetic peptide
SNLYNR

SEQ ID NO: 32 - protein, 6 aa, Artificial Sequence
  Description of Artificial Sequence Synthetic peptide
SNWFNR

SEQ ID NO: 33 - protein, 7 aa, Artificial Sequence
  Description of Artificial Sequence Synthetic peptide
CISKWYL

SEQ ID NO: 34 - protein, 7 aa, Artificial Sequence
  Description of Artificial Sequence Synthetic peptide
CISKLFL

SEQ ID NO: 35 - protein, 7 aa, Artificial Sequence
  Description of Artificial Sequence Synthetic peptide
CISKLYL

SEQ ID NO: 36 - protein, 7 aa, Artificial Sequence
  Description of Artificial Sequence Synthetic peptide
CISKWFL

SEQ ID NO: 37 - protein, 8 aa, Artificial Sequence
  Description of Artificial Sequence Synthetic peptide
LVASSWYY

SEQ ID NO: 38 - protein, 8 aa, Artificial Sequence
  Description of Artificial Sequence Synthetic peptide
LVASSLFY

SEQ ID NO: 39 - protein, 8 aa, Artificial Sequence
  Description of Artificial Sequence Synthetic peptide
LVASSLYY

SEQ ID NO: 40 - protein, 8 aa, Artificial Sequence
  Description of Artificial Sequence Synthetic peptide
LVASSWFY

SEQ ID NO: 41 - protein, 5 aa, Unknown
  Description of Unknown Non-native Enterokinase cleavage site peptide
DDDDK

SEQ ID NO: 42 - protein, 4 aa, Unknown
  Description of Unknown Non-native Factor Xa cleavage site peptide
IEGR

SEQ ID NO: 43 - protein, 4 aa, Unknown
  Description of Unknown Non-native Factor Xa cleavage site peptide
IDGR

SEQ ID NO: 44 - protein, 7 aa, Unknown
  Description of Unknown Non-native Tobacco Etch virus cleavage site peptide
ENLYFQG

SEQ ID NO: 45 - protein, 6 aa, Unknown
  Description of Unknown Non-native Thrombin cleavage site peptide
LVPRGS

SEQ ID NO: 46 - protein, 8 aa, Artificial Sequence
  Description of Artificial Sequence Synthetic peptide
LEVLFQGP